(19) United States
(12) Patent Application Publication
(10) Pub. No.: US 2025/0169412 A1
(43) Pub. Date: May 29, 2025

(54) RICE SEQUENCES INVOLVED IN GRAIN WEIGHT UNDER HIGH TEMPERATURE CONDITIONS AND METHODS OF MAKING AND USING

(71) Applicant: NUtech Ventures, Lincoln, NE (US)

(72) Inventors: Harkamal WALIA, Lincoln, NE (US); Larissa IRVIN, Lincoln, NE (US); Jaspreet SANDHU, Lincoln, NE (US)

(21) Appl. No.: 18/840,986

(22) PCT Filed: Mar. 3, 2023
(86) PCT No.: PCT/US2023/063678

§ 371 Date: Aug. 23, 2024

Related U.S. Application Data
(60) Provisional application No. 63/316,195, filed on Mar. 3, 2022.

(51) Int. Cl. A01H 1/00; A01H 1/04; C07K 14/415

(52) U.S. Cl. CPC A01H 1/12; A01H 1/04; C07K 14/415

(57) ABSTRACT

Higher LOG L1 transcript abundance correlates with lower grain weight under HNT stress. This is supported by higher grain weight of log l1 mutants relative to wild type plants under HNT stress. This finding provides a genetic resource to increase rice adaptation to warming nights. This disclosure describes a novel nucleic acid sequence that, when expressed in a plant (e.g., rice), regulates grain weight and grain number. This disclosure also describes mutant plants and transgenic plants.

### CROSS-REFERENCE TO RELATED APPLICATION

This application claims the benefit of U.S. Application No. 63/316,195 filed Mar. 3, 2022.

### FEDERALLY SPONSORED RESEARCH OR DEVELOPMENT

This invention was made with government support under OIA1736192 awarded by National Science Foundation. The government has certain rights in the invention.

### TECHNICAL FIELD

This disclosure generally relates to nucleic acid sequences encoding polypeptides that are involved in rice grain weight under high temperature conditions.

### BACKGROUND

Global nighttime temperatures are rising at twice the rate than daytime temperatures and pose a challenge to rice (Oryza Sativa) productivity. High night-time temperature (HNT) stress limits rice yield by reducing grain quality, weight and size/fertility. The genetic basis of grain weight regulation under HNT stress remains unknown.

Rice germplasm was examined for the single grain weight (SGW) trait under terminal HNT stress, and three major loci were identified that cumulatively explain 27.8% of the grain weight variation. It was demonstrated that Lonely Guy-like 1 (LOG L1), which encodes for a putative cytokinin activation enzyme, contributes to this variation. Higher LOG L1 transcript abundance correlates with lower grain weight under HNT stress. This is supported by higher grain weight of log l1 mutants relative to wild type plants under HNT stress. This finding provides a genetic resource to increase rice adaptation to warming nights.

### SUMMARY

In one aspect, an isolated nucleic acid molecule is provided, where the nucleic acid molecule has at least 95% sequence identity to SEQ ID NO:1. In some embodiments, the nucleic acid molecule has at least 99% sequence identity to SEQ ID NO: 1. In some embodiments, the nucleic acid molecule is SEQ ID NO:1.

In another aspect, transgenic rice plants transformed with the nucleic acid molecule of any of the preceding claims are provided. In some embodiments, the nucleic acid molecule is operably linked to a promoter functional in rice plants.

In still another aspect, rice plants, or parts thereof, are provided that include a genomic mutation in an endogenous nucleic acid molecule having at least 95% sequence identity to SEQ ID NO:1 and encoding a polypeptide, wherein the genomic mutation confers reduced expression of the endogenous nucleic acid molecule. In some embodiments, the nucleic acid molecule has at least 99% sequence identity to SEQ ID NO:1. In some embodiments, the nucleic acid molecule is SEQ ID NO: 1. In some embodiments, the genomic mutation comprises an insertion, a deletion or a substitution.

In yet another aspect, methods of making a mutant rice plant are provided. Such methods generally include the steps of: a) inducing mutagenesis in rice cells; b) obtaining one or more plants from the cells; and c) identifying at least one of the plants that contains a mutation in a gene having a wild-type sequence as set forth in SEQ ID NO:1 and encoding a polypeptide that regulates grain weight and/or grain number per plant, where the at least one of the plants that contains the mutation exhibits increased grain weight and/or grain number per plant.

In some embodiments, the rice cells are in a seed. In some embodiments, the method further includes the steps of d) crossing the at least one of the plants that contains the mutation with a second rice plant; and e) selecting progeny of the cross that have the at least one mutation, wherein the progeny plant is homozygous for the at least one mutation. In some embodiments, the method further includes the steps of collecting seed produced by the at least one progeny rice plant. In some embodiments, the method further includes the step of growing a rice plant from the at least one progeny plant from the seed.

In another aspect, methods for producing a rice plant are provided. Such methods generally include the steps of: a) providing a first rice plant and a second rice plant, the first rice plant having a mutation in an endogenous nucleic acid sequence having a wild-type sequence as set forth in SEQ ID NO:1 and encoding a polypeptide that regulates grain weight and/or grain number per plant, wherein the first plant exhibits higher grain weight under nighttime or daytime temperature stress, wherein the second plant contains a desired phenotypic trait; b) crossing the first rice plant with the second rice plant to produce one or more F1 progeny plants; c) collecting seed produced by the F1 progeny plants; and d) germinating the seed to produce rice plants exhibiting higher grain weight under nighttime or daytime temperature stress.

In some embodiments, said desired phenotypic trait is selected from the group consisting of disease resistance; high yield; mechanical harvestability; maturation; and grain number per plant. In some embodiments, the method further includes the steps of collecting seed produced by the at least one progeny plant. In some embodiments, the method further includes the steps of growing a plant from the at least one progeny plant from the seed.

In still another aspect, rice plants, or parts thereof, are provided that include a recombinant nucleic acid comprising a heterologous promoter operably linked to a nucleic acid molecule having at least 95% sequence identity to SEQ ID NO:1 or a portion thereof that encodes a polypeptide that regulates grain weight and/or grain number per plant. In some embodiments, the nucleic acid molecule has at least 99% sequence identity to SEQ ID NO:1. In some embodiments, the nucleic acid molecule is SEQ ID NO:1.

In still another aspect, methods of producing a transgenic rice plant are provided. Generally, such methods include: (a) transforming a plurality of plant cells with a plant transformation vector comprising a nucleic acid molecule described herein, wherein the nucleic acid molecule optionally is operably linked to a promoter functional in the plant; (b) regenerating a plurality of transformed plants from the transformed plant cells, and (c) identifying at least one of the transformed plants expressing the nucleic acid.

In some embodiments, the nucleic acid molecule is in an antisense orientation. In some embodiments, the nucleic acid molecule is in a sense orientation. In some embodiments, the nucleic acid molecule is expressed as a double stranded RNA molecule. In some embodiments, the plant cells are in a seed.

In one aspect, methods are provided that include the steps of: a) obtaining a rice plant comprising a recombinant nucleic acid molecule that includes a nucleic acid molecule as described herein, wherein the nucleic acid molecule optionally is operably linked to a promoter functional in the plant; b) crossing the plant with a second plant lacking the recombinant nucleic acid molecule; and c) producing at least one progeny plant from the crossing, wherein the progeny plants comprise the nucleic acid molecule.

In some embodiments, the nucleic acid molecule is in an antisense orientation. In some embodiments, the nucleic acid molecule is in a sense orientation. In some embodiments, the nucleic acid molecule is expressed as a double stranded RNA molecule.

In one aspect, an isolated nucleic acid molecule is provided, where the nucleic acid molecule has at least 95% sequence identity to SEQ ID NO:3. In some embodiments, the nucleic acid molecule has at least 99% sequence identity to SEQ ID NO:3. In some embodiments, the nucleic acid molecule is SEQ ID NO:3.

In another aspect, transgenic rice plants transformed with the nucleic acid molecule of any of the preceding claims are provided. In some embodiments, the nucleic acid molecule is operably linked to a promoter functional in rice plants.

In still another aspect, rice plants, or parts thereof, are provided that include a genomic mutation in an endogenous nucleic acid molecule having at least 95% sequence identity to SEQ ID NO:3 and encoding a polypeptide, wherein the genomic mutation confers reduced expression of the endogenous nucleic acid molecule. In some embodiments, the nucleic acid molecule has at least 99% sequence identity to SEQ ID NO:3. In some embodiments, the nucleic acid molecule is SEQ ID NO:3. In some embodiments, the genomic mutation comprises an insertion, a deletion or a substitution.

In yet another aspect, methods of making a mutant rice plant are provided. Such methods generally include the steps of: a) inducing mutagenesis in rice cells; b) obtaining one or more plants from the cells; and c) identifying at least one of the plants that contains a mutation in a gene having a wild-type sequence as set forth in SEQ ID NO:3 and encoding a polypeptide that regulates grain weight and/or grain number per plant, where the at least one of the plants that contains the mutation exhibits increased grain weight and/or grain number per plant.

In some embodiments, the rice cells are in a seed. In some embodiments, the method further includes the steps of d) crossing the at least one of the plants that contains the mutation with a second rice plant; and e) selecting progeny of the cross that have the at least one mutation, wherein the progeny plant is homozygous for the at least one mutation. In some embodiments, the method further includes the steps of collecting seed produced by the at least one progeny rice plant. In some embodiments, the method further includes the step of growing a rice plant from the at least one progeny plant from the seed.

In another aspect, methods for producing a rice plant are provided. Such methods generally include the steps of: a) providing a first rice plant and a second rice plant, the first rice plant having a mutation in an endogenous nucleic acid sequence having a wild-type sequence as set forth in SEQ ID NO:3 and encoding a polypeptide that regulates grain weight and/or grain number per plant, wherein the first plant exhibits higher grain weight under nighttime or daytime temperature stress, wherein the second plant contains a desired phenotypic trait; b) crossing the first rice plant with the second rice plant to produce one or more F1 progeny plants; c) collecting seed produced by the F1 progeny plants; and d) germinating the seed to produce rice plants exhibiting higher grain weight under nighttime or daytime temperature stress.

In some embodiments, said desired phenotypic trait is selected from the group consisting of disease resistance; high yield; mechanical harvestability; maturation; and grain number per plant. In some embodiments, the method further includes the steps of collecting seed produced by the at least one progeny plant. In some embodiments, the method further includes the steps of growing a plant from the at least one progeny plant from the seed.

In still another aspect, rice plants, or parts thereof, are provided that include a recombinant nucleic acid comprising a heterologous promoter operably linked to a nucleic acid molecule having at least 95% sequence identity to SEQ ID NO:3 or a portion thereof that encodes a polypeptide that regulates grain weight and/or grain number per plant. In some embodiments, the nucleic acid molecule has at least 99% sequence identity to SEQ ID NO:3. In some embodiments, the nucleic acid molecule is SEQ ID NO:3.

In still another aspect, methods of producing a transgenic rice plant are provided. Generally, such methods include: (a) transforming a plurality of plant cells with a plant transformation vector comprising a nucleic acid molecule described herein, wherein the nucleic acid molecule optionally is operably linked to a promoter functional in the plant; (b) regenerating a plurality of transformed plants from the transformed plant cells, and (c) identifying at least one of the transformed plants expressing the nucleic acid.

In some embodiments, the nucleic acid molecule is in an antisense orientation. In some embodiments, the nucleic acid molecule is in a sense orientation. In some embodiments, the nucleic acid molecule is expressed as a double stranded RNA molecule. In some embodiments, the plant cells are in a seed.

In one aspect, methods are provided that include the steps of: a) obtaining a rice plant comprising a recombinant nucleic acid molecule that includes a nucleic acid molecule as described herein, wherein the nucleic acid molecule optionally is operably linked to a promoter functional in the plant; b) crossing the plant with a second plant lacking the recombinant nucleic acid molecule; and c) producing at least one progeny plant from the crossing, wherein the progeny plants comprise the nucleic acid molecule.

In some embodiments, the nucleic acid molecule is in an antisense orientation. In some embodiments, the nucleic acid molecule is in a sense orientation. In some embodiments, the nucleic acid molecule is expressed as a double stranded RNA molecule.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1A-1F demonstrate genome-wide association (GWA) analysis for single grain weight (SGW) under control (C) and high night-time temperature (HNT) conditions for rice. Rice diversity panel 1 (RDP1) accessions were exposed to C or HNT treatment beginning at 1 d after fertilization till maturity. The mature grains were used to measure SGW. FIG. 1A shows natural variation in SGW (mean) of RDP1 accessions under control and HNT conditions. Genotypes (on x-axis) are ordered based on the percentage change in SGW of HNT-treated seeds relative to control seeds. FIG. 1B shows Manhattan plots from GWA analysis for SGW under C and HNT. Significant (P<3.3 Å˜10−6 or −log 10(P)>5.4) single nucleotide polymorphisms (SNPs) are above threshold (blue line). X-axis represent chromosome numbers. Blue arrow indicates most significant SNP for SGW1 (qSGW1; ID, SNP-1.29438503; Position, Chr1:29439549) for HNT. qSGW1 is represented by maroon dot in control and HNT Manhattan plots. Previously known genes/quantitative trait loci (QTLs) (TGW6, RBG1, OsSWEETI4) co-localized with GWAS peaks. FIG. 1C is a zoom-in plot showing two most-significant peaks on chromosome 1. HNT-specific topmost-significant SNP (qSGW1) localized to first intron of a candidate gene, Os01g51210 (Lonely Guy Like 1, LOG L1). FIG. 1D is a box-plot showing allelic effect of qSGW1 on SGW in RDP1. Here, heavy-grain (HG) and light-grain (LG) represent two allelic groups at qSGW1. p-values (indicated by text) represent significant difference between SGW of HG and LG allelic groups. FIG. 1E show the subpopulation level distribution of HGA and LGA alleles among RDP1 accessions. Box plots showing impact of stacking the favorable alleles of the lead HNT-specific SNP (qSGW1) and common (detected under both control and HNT) SNPs on Chr 6 (qSGW6) on SGW under HNT. Here, ATT, ACT, ACC, and CCC represents four different haplotypes (hap) groups. The text in parenthesis indicates number of favorable alleles in each hap group.

FIG. 2A-2D demonstrate that LOG1 overexpression reduces yield parameters and increases HNT sensitivity of SGW. Florets were marked at time of flowering, and at 1 d after flowering (DAF), wild-type (WT), over-expression (OE) and CRISPR-Cas9 knock-out (KO) plants were exposed to either control (C) or high night temperature (HNT) treatment until physiological maturity. FIG. 2A shows mature marked grains were used for collecting SGW (FIG. 2A) and grain thickness (FIG. 2B). Whole plant data was collected by measuring seed number (FIG. 2C) and yield per plant (FIG. 2D). Box plot represents range, median and mean (filled circle) for nine plants. Here, (*, P-value <0.1; **, P-value <0.01; ***, P-value <0.001; ****, P-value <0.0001). T-test was used to compare KO and OE to WT within C (blue asterisks) or HNT (red asterisks) as well as for comparing HNT to control (black asterisks) within a genotype. Significant difference (t-test) between C and HNT within each genotype is indicated by black asterisks. In FIGS. 2A, 2C, and 2D, text (%) represent percentage difference in SGW for comparisons between different groups as indicated by dotted lines.

FIG. 3 shows the natural variation in single grain weight (SGW) and high-night temperature (HNT) response of fertility among RDP1 accessions. Blue and red bars represents SGW under control (C) and HNT conditions, respectively. Dotted yellow lines represents 5% cutoff for percentage change in fertility of HNT-treated plants compered to control (on right Y-axis, represented by black dots).

FIG. 4 shows the haploview of r² between significant SNPs on chromosome 1.

FIG. 5 is a box-plot showing allelic effect of qSGW1 on SGW in different subpopulations of RDP1. P-values (indicated by text) were calculated by comparing allelic groups within a treatment. Heavy-grain (HG) and light-grain (LG) represent two allelic groups at qSGW1.

FIG. 6 are box-plots showing allelic effect of three major loci, qSGW1, sSGW6.1, and sSGW6.2 on SGW.

FIG. 7 shows the impact of stacking favorable alleles for three major peaks on SGW under control and HNT. Means with same significance letter are not significantly different from each other (pairwise t-test).

FIG. 8 shows that the protein coding genes in the vicinity of qSGW1 were evaluated for their expression in different rice developmental stages and tissues. The ten genes with high expression near flowering time are highlighted in green.

FIG. 9 show that the selected 10 genes that have preferentially higher expression in inflorescence or grains during flowering phase were evaluated for the allelic variations at transcript level. For this, the natural variation in expression of these 10 genes were explored and it was found that LOG L1 showed about 2-fold higher transcript abundance in LG than HG allelic group accessions. FC: fold change.

FIG. 10A-10C shows LOG L1 CRISPR-Cas9 based knockout (KO) and overexpression. FIG. 10A shows the gene structure and positions (red) of guide RNA 1 (gR1) and gR2. The corresponding KOs obtained from each of gR are indicated with black text. FIG. 10B shows the Sanger sequencing-based sequence analysis of homozygous KO mutants. Text in parenthesis represents type of mutation. FIG. 10C shows the relative transcript abundance (mean±SD) of LOG L1 at two days after fertilization. Error bars represent ±SD.

FIG. 11A-11B shows that LOG1 KOs have increased grain size under control and HNT. Florets were marked at time of flowering, and at 1 d after flowering (DAF), wild-type (WT), over-expression (OE) and CRISPR-Cas9 knock-out (KO) plants were exposed to either control (C) or high night temperature (HNT) treatment until physiological maturity. FIG. 11A shows that mature marked grains were used for collecting grain size parameters. FIG. 11B and FIG. 11C are box plots that represents range, median and mean (filled circle) for nine plants that represent grain length and grain width, respectively. T-test was used to compare HNT to control (black text) within a genotype. Significant difference (t-test) between C and HNT within each genotype is indicated by p-values.

FIG. 12 shows that LOG1 overexpression reduces tiller number and increases grain size under control and HNT. Number of tillers for KO, OE and WT in one month and flowering plants. T-test was used to compare KO and OE genotypes to WT and text on box plots represents p-values. n=24.

### DETAILED DESCRIPTION

Novel nucleic acids are provided herein (see, for example, SEQ ID NO: 1). As used herein, nucleic acids can include DNA and RNA, and includes nucleic acids that contain one or more nucleotide analogs or backbone modifications. A nucleic acid can be single stranded or double stranded, which usually depends upon its intended use. The nucleic acid provided herein encodes a polypeptide having the sequence shown in SEQ ID NO:2.

LOG1 from rice
(SEQ ID NO: 1)
ATGGGCGACAACAGCGCCGCCGCGGCGGCCGTGGCCGCGCCGCGCGGCAGGTTCGGCAGGATCTGCGTCTT
CTGCGGCAGCAACGCCGGCAACCGCGCGGTGTTCGGCGACGCGGCGCTCCAGCTCGGGCAGGAGCTGGTGT
CGAGAGGGATCGAGTTGGTCTACGGTGGCGGCAGCGTCGGGTTGATGGGCTTGATCGCGCAGACGGTTCTT
GATGGCGGCTGCGGTGTTCTCGGGGTGATTCCAAAAGCACTCATGCCCACCGAGATATCAGGTGCAAGTGT
TGGAGAAGTGAAAATTGTGTCTGACATGCATGAGAGGAAAGCTGAGATGGCACGCCAATCCGATGCCTTCA
TCGCTCTTCCTGGAGGGTATGGAACAATGGAGGAGTTGTTAGAGATGATAACTTGGTCACAACTTGGAATT
CATGACAAACCAGTTGGGTTGCTGAATGTGGACGGTTACTATGATCCGTTGCTTGCGCTATTTGATAAGGG
TGCGGCAGAAGGATTTATTAAGGCCGATTGCAGACAAATAATTGTTTCGGCACCGACTGCGCATGAGCTGC
TGAGAAAGATGGAGCAATACACTCGTTCACACCAGGAGGTAGCGCCACGTACAAGCTGGGAGATGTCAGAG
CTTGGTTATGGAAAGACACCAGAGGAATCGTAG
(SEQ ID NO: 2)
MGDNSAAAAAVAAPRGRFGRICVFCGSNAGNRAVFGDAALQLGQELVSRGIELVYGGGSVGLMGLIAQTVL
DGGCGVLGVIPKALMPTEISGASVGEVKIVSDMHERKAEMARQSDAFIALPGGYGTMEELLEMITWSQLGI
HDKPVGLLNVDGYYDPLLALFDKGAAEGFIKADCRQIIVSAPTAHELLRKMEQYTRSHQEVAPRISWEMSE
LGYGKTPEES

In addition to the nucleic acids and polypeptides disclosed herein (i.e., SEQ ID NOs: 1 and 2), the skilled artisan will further appreciate that changes can be introduced into a nucleic acid molecule (e.g., SEQ ID NO:1), thereby leading to changes in the amino acid sequence of the encoded polypeptide (e.g., SEQ ID NO:2). For example, changes can be introduced into nucleic acid coding sequences using mutagenesis (e.g., site-directed mutagenesis, PCR-mediated mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al. (1978, in Atlas of Protein Sequence and Structure, 5(Suppl. 3):345-352), which provides frequency tables for amino acid substitutions), and a non-conservative substitution is one in which an amino acid residue is replaced with an amino acid residue that does not have a similar side chain.

In addition to SEQ ID NO:1 and 2, nucleic acids and polypeptides are provided that differ from SEQ ID NO:1 and 2, respectively. Nucleic acids and polypeptides that differ in sequence from SEQ ID NO:1 and 2 can have at least 80% sequence identity (e.g., at least 85%, 90%, 95%, or 99% sequence identity) to SEQ ID NO:1 and 2, respectively. Similarly, nucleic acids and polypeptides are provided that differ from SEQ ID NO:3 and 4, respectively. Nucleic acids and polypeptides that differ in sequence from SEQ ID NO:3 and 4 can have at least 80% sequence identity (e.g., at least 85%, 90%, 95%, or 99% sequence identity) to SEQ ID NO:3 and 4, respectively.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the algorithm described by Altschul et al. (1997, Nucleic Acids Res., 25:3389 3402) as incorporated into BLAST (Basic Local Alignment Search Tool) programs, available at ncbi.nlm.nih.gov on the World Wide Web. BLASTN is the program used to align and compare the identity between nucleic acid sequences, while BLASTP is the program used to align and compare the identity between amino acid sequences. When utilizing BLAST programs to calculate the percent identity between a sequence and another sequence, the default parameters of the respective programs generally are used.

Nucleic acid fragments are included in the invention. Nucleic acid fragments suitable for use in the invention are those fragments that encode a polypeptide having functional activity. These fragments can be called “functional fragments,” although it is understood that it is not the nucleic acid that possesses functionality.

As used herein, an “isolated” nucleic acid molecule is a nucleic acid molecule that is free of sequences that naturally flank one or both ends of the nucleic acid in the genome of the organism from which the isolated nucleic acid molecule is derived (e.g., a cDNA or genomic DNA fragment produced by PCR or restriction endonuclease digestion). Such an isolated nucleic acid molecule is generally introduced into a vector (e.g., a cloning vector, or an expression vector) for convenience of manipulation or to generate a fusion nucleic acid molecule, discussed in more detail below. In addition, an isolated nucleic acid molecule can include an engineered nucleic acid molecule such as a recombinant or a synthetic nucleic acid molecule.

As used herein, a “purified” polypeptide is a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the proteins and naturally occurring molecules with which it is naturally associated. Since a polypeptide that is chemically synthesized is, by nature, separated from the components that naturally accompany it, a synthetic polypeptide is “purified.”

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

A vector containing a nucleic acid (e.g., a nucleic acid that encodes a polypeptide) also is provided. Vectors, including expression vectors, are commercially available or can be produced by recombinant DNA techniques routine in the art. A vector containing a nucleic acid can have expression elements operably linked to such a nucleic acid, and further can include sequences such as those encoding a selectable marker (e.g., an antibiotic resistance gene). A vector containing a nucleic acid can encode a chimeric or fusion polypeptide (i.e., a polypeptide operatively linked to a heterologous polypeptide, which can be at either the N-terminus or C-terminus of the polypeptide). Representative heterologous polypeptides are those that can be used in purification of the encoded polypeptide (e.g., 6×His tag, glutathione S-transferase (GST))

Expression elements include nucleic acid sequences that direct and regulate expression of nucleic acid coding sequences. One example of an expression element is a promoter sequence. Expression elements also can include introns, enhancer sequences, response elements, or inducible elements that modulate expression of a nucleic acid. Expression elements can be of bacterial, yeast, insect, mammalian, or viral origin, and vectors can contain a combination of elements from different origins. As used herein, operably linked means that a promoter or other expression element(s) are positioned in a vector relative to a nucleic acid in such a way as to direct or regulate expression of the nucleic acid (e.g., in-frame). Many methods for introducing nucleic acids into host cells, both in vivo and in vitro, are well known to those skilled in the art and include, without limitation, electroporation, calcium phosphate precipitation, polyethylene glycol (PEG) transformation, heat shock, lipofection, microinjection, and viral-mediated nucleic acid transfer.

Vectors as described herein can be introduced into a host cell. As used herein, “host cell” refers to the particular cell into which the nucleic acid is introduced and also includes the progeny or potential progeny of such a cell. A host cell can be any prokaryotic or eukaryotic cell. For example, nucleic acids can be expressed in bacterial cells such as E. coli, or in insect cells, yeast or mammalian cells (such as Chinese hamster ovary cells (CHO) or COS cells). Other suitable host cells are known to those skilled in the art.

Nucleic acids can be detected using any number of amplification techniques (see, e.g., PCR Primer: A Laboratory Manual, 1995, Dieffenbach & Dveksler, Eds., Cold Spring Harbor Laboratory Press, Cold Spring Harbor, NY; and U.S. Pat. Nos. 4,683,195; 4,683,202; 4,800,159; and 4,965,188) with an appropriate pair of oligonucleotides (e.g., primers). A number of modifications to the original PCR have been developed and can be used to detect a nucleic acid.

Nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY; Sections 7.37-7.57, 9.47-9.57, 11.7-11.8, and 11.45-11.57). Sambrook et al. discloses suitable Southern blot conditions for oligonucleotide probes less than about 100 nucleotides (Sections 11.45-11.46). The Tm between a sequence that is less than 100 nucleotides in length and a second sequence can be calculated using the formula provided in Section 11.46. Sambrook et al. additionally discloses Southern blot conditions for oligonucleotide probes greater than about 100 nucleotides (see Sections 9.47-9.54). The Tm between a sequence greater than 100 nucleotides in length and a second sequence can be calculated using the formula provided in Sections 9.50-9.51 of Sambrook et al.

The conditions under which membranes containing nucleic acids are prehybridized and hybridized, as well as the conditions under which membranes containing nucleic acids are washed to remove excess and non-specifically bound probe, can play a significant role in the stringency of the hybridization. Such hybridizations and washes can be performed, where appropriate, under moderate or high stringency conditions. For example, washing conditions can be made more stringent by decreasing the salt concentration in the wash solutions and/or by increasing the temperature at which the washes are performed. For example, stringent salt concentration typically is less than about 750 mM NaCl and 75 mM trisodium citrate (e.g., less than about 500 mM NaCl and 50 mM trisodium citrate; less than about 250 mM NaCl and 25 mM trisodium citrate). High stringency hybridization can be obtained in the presence of at least about 35% formamide (e.g., at least about 50% formamide). Stringent temperature conditions will ordinarily include temperatures of at least about 30° C. (e.g., at least about 37° C., at least about 42° C.). Varying additional parameters, such as hybridization time, the concentration of detergent (e.g., sodium dodecyl sulfate (SDS)), and the inclusion or exclusion of carrier DNA, are well known to those skilled in the art. Various levels of stringency are accomplished by combining these various conditions as needed. In some embodiments, hybridization occurs at 30° C. in 750 mM NaC, 75 mM trisodium citrate, and 1% SDS. In some embodiments, hybridization occurs at 37° C. in 500 mM NaC, 50 mM trisodium citrate, 1% SDS, 35% formamide, and 100 μg/ml denatured salmon sperm DNA (ssDNA). In some embodiments, hybridization occurs at 42° C. in 250 mM NaCl, 25 mM trisodium citrate, 1% SDS, 50% formamide, and 200 μg/ml ssDNA. Useful variations on these conditions will be readily apparent to those skilled in the art.

In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium. It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane.

A nucleic acid molecule is deemed to hybridize to a nucleic acid but not to another nucleic acid if hybridization to a nucleic acid is at least 5-fold (e.g., at least 6-fold, 7-fold, 8-fold, 9-fold, 10-fold, 20-fold, 50-fold, or 100-fold) greater than hybridization to another nucleic acid. The amount of hybridization can be quantitated directly on a membrane or from an autoradiograph using, for example, a PhosphorImager or a Densitometer (Molecular Dynamics, Sunnyvale, CA).

Polypeptides can be detected using antibodies. Techniques for detecting polypeptides using antibodies include enzyme linked immunosorbent assays (ELISAs), Western blots, immunoprecipitations and immunofluorescence. An antibody can be polyclonal or monoclonal. An antibody having specific binding affinity for a polypeptide can be generated using methods well known in the art. The antibody can be attached to a solid support such as a microtiter plate using methods known in the art. In the presence of a polypeptide, an antibody-polypeptide complex is formed.

Detection (e.g., of an amplification product, a hybridization complex, or a polypeptide) is usually accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

Plant varieties, lines, or cultivars are provided that have a mutation in the endogenous nucleic acid described herein (e.g., SEQ ID NO:1 or 3). As described herein, plants having a mutation in the endogenous nucleic acid (e.g., SEQ ID NO:1 or 3) can exhibit an increase in grain weight and/or grain number per plant, e.g., under temperature stress, compared to a corresponding plant lacking the mutation and grown under corresponding conditions. In addition, plants having a mutation in the endogenous nucleic acid (e.g., SEQ ID NO:1 or 3) can exhibit an increase in grain weight and/or grain number per plant, e.g., under temperature stress, compared to a corresponding plant lacking the mutation and grown under corresponding conditions.

Methods of making a plant having a mutation are known in the art. Mutations can be random mutations or targeted mutations. For random mutagenesis, plant cells can be mutagenized using, for example, a chemical mutagen, ionizing radiation, or fast neutron bombardment (see, e.g., Li et al., 2001, Plant J., 27:235-42). Representative chemical mutagens include, without limitation, nitrous acid, sodium azide, acridine orange, ethidium bromide, and ethyl methane sulfonate (EMS), while representative ionizing radiation includes, without limitation, x-rays, gamma rays, fast neutron irradiation, and UV irradiation. The dosage of the mutagenic chemical or radiation is determined experimentally for each type of plant tissue such that a mutation frequency is obtained that is below a threshold level characterized by lethality or reproductive sterility. The number of M₁ generation seed or the size of M₁ plant populations resulting from the mutagenic treatments are estimated based on the expected frequency of mutations. For targeted mutagenesis, representative technologies include TALEN technology (see, for example, Li et al., 2011, Nucleic Acids Res., 39(14):6315-25), zinc-finger technology (see, for example, Wright et al., 2005, The Plant J, 44:693-705), and CRISPR technology (see, for example, Mali et al., 2013, Nature Methods, 10:957-63). Whether random or targeted, a mutation can be a point mutation, an insertion, a deletion, a substitution, or combinations thereof.

As discussed herein, one or more nucleotides can be mutated to alter the expression and/or function of the encoded polypeptide, relative to the expression and/or function of the corresponding wild type polypeptide. It will be appreciated, for example, that a mutation in one or more of the highly conserved regions would likely alter polypeptide function, while a mutation outside of those conserved regions may have little to no effect on polypeptide function. In addition, a mutation in a single nucleotide can create a stop codon, which would result in a truncated polypeptide and, depending on the extent of truncation, loss-of-function.

A mutation in one of the nucleic acids disclosed herein results in reduced or even complete elimination of LOG1 and/or LOG7 expression and/or activity in a plant comprising the mutation. Suitable types of mutations include, without limitation, insertions of nucleotides, deletions of nucleotides, or transitions or transversions. In some instances, a mutation is a point mutation; in some instances, a mutation encompasses multiple nucleotides. In some cases, a sequence includes more than one mutation or more than one type of mutation.

For example, a mutation in a promoter sequence can result in reduced or complete elimination of LOG1 and/or LOG7 expression in a plant comprising the mutation. For example, a mutation in a promoter sequence can alter or eliminate the binding or recognition site of a transcription factor or of the polymerase enzyme, or a mutation in a promoter sequence can alter or eliminate the function of an enhancer, an activator or the like, or a repressor, a silencer or the like. Mutations in a promoter sequence can result in altered or absent transcription, or production of a less-than-functional or non-functional transcript. A less-than-functional or non-functional transcript can result from improper expression (e.g., expressed in the wrong place or at the wrong time), or from degradation of the transcript. Alternatively, a mutation in a promoter sequence may allow transcription to take place, but may interfere with or eliminate the ability of the transcript to be translated.

Mutations in a coding sequence can result in insertions of one or more amino acids, deletions of one or more amino acids, and/or non-conservative amino acid substitutions in the encoded polypeptide. Insertion or deletion of amino acids in a coding sequence, for example, can disrupt the conformation of the encoded polypeptide. Amino acid insertions or deletions also can disrupt sites important for recognition of a binding ligand or for activity of the polypeptide. It is known in the art that the insertion or deletion of a larger number of contiguous amino acids is more likely to render the gene product non-functional, compared to a smaller number of inserted or deleted amino acids. In addition, one or more mutations can change the localization of a polypeptide, introduce a stop codon to produce a truncated polypeptide, or disrupt an active site or domain (e.g., a catalytic site or domain, a binding site or domain) within the polypeptide.

Non-conservative amino acid substitutions can replace an amino acid of one class with an amino acid of a different class. Non-conservative substitutions can make a substantial change in the charge or hydrophobicity of the gene product. Non-conservative amino acid substitutions can also make a substantial change in the bulk of the residue side chain, e.g., substituting an alanine residue for an isoleucine residue. Examples of non-conservative substitutions include a basic amino acid for a non-polar amino acid, or a polar amino acid for an acidic amino acid.

Polypeptides can include particular sequences that determine where the polypeptide is located within the cell, within the membrane, or outside of the cell. Target peptide sequences often are cleaved (e.g., by specific proteases that recognize a specific nucleotide motif) after the polypeptide is localized to the appropriate position. By mutating the target sequence or a cleavage motif, the location of the polypeptide can be altered.

It would be understood by a skilled artisan that mutations also can include larger mutations such as, for example, deletion of most or all of the promoter, deletion of most of all of the coding sequence, or deletion or translocation of the chromosomal region containing some or all of the LOG1 and/or LOG7 sequences. It would be understood, however, that, the larger the mutation, the more likely it is to have an effect on other traits as well.

Following mutagenesis, M₀ plants are regenerated from the mutagenized cells and those plants, or a subsequent generation of that population (e.g., M₁, M₂, M₃, etc.), can be screened for a mutation in SEQ ID NO:1 or 3. Screening for plants carrying a mutation in a sequence of interest can be performed using methods routine in the art (e.g., hybridization, amplification, combinations thereof) or by evaluating the phenotype of the plants (e.g., an increase in grain weight and/or grain number per plant, e.g., under temperature stress). Generally, the presence of a mutation in the nucleic acid sequence disclosed herein (e.g., SEQ ID NO:1 or 3) results in an increase in grain weight and/or grain number per plant, e.g., under temperature stress, compared to a corresponding plant (e.g., having the same varietal background) lacking the mutation under corresponding growth conditions.

As used herein, an “increase” in grain weight and/or grain number per plant, e.g., under temperature stress, refers to an increase (e.g., a statistically significant increase) in the indicated feature under the indicated temperature condition by at least about 5% up to about 95% (e.g., about 5% to about 10%, about 5% to about 20%, about 5% to about 50%, about 5% to about 75%, about 10% to about 25%, about 10% to about 50%, about 10% to about 90%, about 20% to about 40%, about 20% to about 60%, about 20% to about 80%, about 25% to about 75%, about 50% to about 75%, about 50% to about 85%, about 50% to about 95%, and about 75% to about 95%) relative to the same feature from a corresponding plant lacking the mutation grown under corresponding conditions. As used herein, statistical significance refers to a p-value of less than 0.05, e.g., a p-value of less than 0.025 or a p-value of less than 0.01, using an appropriate measure of statistical significance, e.g., a one-tailed two sample t-test.

An M₁ plant may be heterozygous for a mutant allele and exhibit a wild type phenotype. In such cases, at least a portion of the first generation of self-pollinated progeny of such a plant exhibits a wild type phenotype. Alternatively, an Mt plant may have a mutant allele and exhibit a mutant phenotype. Such plants may be heterozygous and exhibit a mutant phenotype due to a phenomenon such as dominant negative suppression, despite the presence of the wild type allele, or such plants may be homozygous due to independently induced mutations in both alleles.

A plant carrying a mutant allele can be used in a plant breeding program to create novel and useful cultivars, lines, varieties and hybrids. Thus, in some embodiments, an M₁, M₂, M₃ or later generation plant containing at least one mutation is crossed with a second plant, and progeny of the cross are identified in which the mutation(s) is present. It will be appreciated that the second plant can contain the same mutation as the plant to which it is crossed, a different mutation, or be wild type at the locus. Additionally or alternatively, a second line can exhibit a phenotypic trait such as, for example, disease resistance; high yield; mechanical harvestability; maturation; and grain number per plant.

Breeding can be carried out using known procedures. DNA fingerprinting, SNP or similar technologies can be used in a marker-assisted selection (MAS) breeding program to transfer or breed mutant alleles into other lines, varieties or cultivars, as described herein. Progeny of the cross can be screened for a mutation using methods described herein, and plants having a mutation in a nucleic acid sequence disclosed herein (e.g., SEQ ID NO:1 or 3) can be selected. For example, plants in the F2 or backcross generations can be screened using a marker developed from a sequence described herein or a fragment thereof, using one of the techniques listed herein. Plants also can be screened for an increase in grain weight and/or grain number per plant, e.g., under temperature stress, and those plants having one or more of such phenotypes, compared to a corresponding plant that lacks the mutation, can be selected. Plants identified as possessing the mutant allele and/or the mutant phenotype can be backcrossed or self-pollinated to create a second population to be screened. Backcrossing or other breeding procedures can be repeated until the desired phenotype of the recurrent parent is recovered.

Successful crosses yield F₁ plants that are fertile and that can be backcrossed with one of the parents if desired. In some embodiments, a plant population in the F₂ generation is screened for the mutation or variant gene expression using standard methods (e.g., PCR with primers based upon the nucleic acid sequences disclosed herein). Selected plants are then crossed with one of the parents and the first backcross (BC₁) generation plants are self-pollinated to produce a BC₁F₂ population that is again screened for variant gene expression. The process of backcrossing, self-pollination, and screening is repeated, for example, at least four times until the final screening produces a plant that is fertile and reasonably similar to the recurrent parent. This plant, if desired, is self-pollinated and the progeny are subsequently screened again to confirm that the plant contains the mutation and exhibits variant gene expression. Breeder's seed of the selected plant can be produced using standard methods including, for example, field testing, genetic analysis, and/or confirmation of the phenotype.

The result of a plant breeding program using the mutant plants described herein are novel and useful cultivars, varieties, and lines. As used herein, the term “variety” refers to a population of plants that share constant characteristics that separate them from other plants of the same species. A variety is often, although not always, sold commercially. While possessing one or more distinctive traits, a variety is further characterized by a very small overall variation between individual with that variety. A “pure line” variety may be created by several generations of self-pollination and selection, or vegetative propagation from a single parent using tissue or cell culture techniques. A “line,” as distinguished from a variety, most often denotes a group of plants used non-commercially, for example, in plant research. A line typically displays little overall variation between individuals for one or more traits of interest, although there may be some variation between individuals for other traits.

Varieties, lines and cultivars described herein can be used to form single-cross F₁ hybrids. In such embodiments, the plants of the parent varieties can be grown as substantially homogeneous adjoining populations to facilitate natural cross-pollination from the male parent plants to the female parent plants. The F₂ seed formed on the female parent plants is selectively harvested by conventional means. One also can grow the two parent plant varieties in bulk and harvest a blend of F₁ hybrid seed formed on the female parent and seed formed upon the male parent as the result of self-pollination. Alternatively, three-way crosses can be carried out wherein a single-cross F₁ hybrid is used as a female parent and is crossed with a different male parent. As another alternative, double-cross hybrids can be created wherein the F₁ progeny of two different single-crosses are themselves crossed. Self-incompatibility can be used to particular advantage to prevent self-pollination of female parents when forming a double-cross hybrid.

In addition to mutation, another way in which LOG1 expression can be reduced or knocked-out is to use inhibitory RNAs (e.g., RNAi). Therefore, transgenic plants are provided that contain a transgene encoding at least one RNAi molecule, which, when expressed, silences the endogenous nucleic acid described herein (e.g., SEQ ID NO:1 or 3). As described herein, such transgenic plants exhibit an increase in grain weight and/or grain number per plant, e.g., under temperature stress (e.g., compared to a plant lacking or not expressing the RNAi).

RNAi technology is known in the art and is a very effective form of post-transcriptional gene silencing. RNAi molecules typically contain a nucleotide sequence (e.g., from about 18 nucleotides in length (e.g., about 19 or 20 nucleotides in length) up to about 700 nucleotides in length) that is complementary to the target gene in both the sense and antisense orientations. The sense and antisense strands can be connected by a short “loop” sequence (e.g., about 5 nucleotides in length up to about 800 nucleotides in length) and expressed in a single transcript, or the sense and antisense strands can be delivered to and expressed in the target cells on separate vectors or constructs. A number of companies offer RNAi design and synthesis services (e.g., Life Technologies, Applied Biosystems).

The RNAi molecule can be expressed using a plant expression vector. The RNAi molecule typically is at least 25 nucleotides in length and has at least 91% sequence identity (e.g., at least 95%, 96%, 97%, 98% or 99% sequence identity) to the nucleic acid sequence disclosed herein (e.g., SEQ ID NO:1 or 3) or hybridizes under stringent conditions to the nucleic acid sequence disclosed herein (e.g., SEQ ID NO:1 or 3). Hybridization under stringent conditions is described above.

Methods of introducing a nucleic acid (e.g., a heterologous nucleic acid) into plant cells are known in the art and include, for example, particle bombardment, Agrobacterium-mediated transformation, microinjection, polyethylene glycol-mediated transformation (e.g., of protoplasts, see, for example, Yoo et al. (2007, Nature Protocols, 2(7):1565-72)), liposome-mediated DNA uptake, or electroporation. Following transformation, the transgenic plant cells can be regenerated into transgenic plants. As described herein, expression of the transgene results in plants that exhibit an increase in grain weight and/or grain number per plant, e.g., under temperature stress, relative to a plant not expressing the transgene. The regenerated transgenic plants can be screened for an increase in grain weight and/or grain number per plant, e.g., under temperature stress, compared to a corresponding non-transgenic plant, and can be selected for use in, for example, a breeding program as discussed herein.

The sequences described herein can be overexpressed in plants, if so desired. Therefore, transgenic plants are provided that are transformed with a nucleic acid molecule described herein (e.g., SEQ ID NO:1 or 3) or a functional fragment thereof under control of a promoter that is able to drive expression in plants (e.g., a plant promoter). As discussed herein, a nucleic acid molecule used in a plant expression vector can have a different sequence than a sequence described herein, which can be expressed as a percent sequence identity (e.g., relative to SEQ ID NO:1 or 3) or based on the conditions under which sequences hybridize (e.g., to SEQ ID NO:1 or 3). As an alternative to using a full-length sequence, a portion of the sequence can be used that encodes a polypeptide fragment having the desired functionality (referred to herein as a “functional fragment”). When used with respect to nucleic acids, it would be appreciated that it is not the nucleic acid fragment that possesses functionality but the encoded polypeptide fragment.

Following transformation, the transgenic cells can be regenerated into transgenic plants, which can be screened for a decrease in grain weight and/or grain number per plant, e.g., under temperature stress, and plants having decreased amounts of at least one of such features, compared to the feature in a corresponding non-transgenic plant, can be selected and used, for example, in a breeding program as discussed herein.

LOG7 is the closest homolog of LOG1 in rice. The nucleic acid coding sequence and protein sequences for LOG7 are provided below in SEQ ID NOs:3 and 4:

LOG7 from rice
(SEQ ID NO: 3)
ATGGGAGACGGAGCGGAGGCGGCGGGGGCGACCGCGGCGAGCAGGTTCGGGACGATCTGCGTCTTCTGCGGCAG
CAACGCGGGGCGCCGCAGGGTGTTCGGCGACGCGGCGCTCGACCTCGGCCACGAGCTGGTGAGGCGGGGCGTCG
ATCTGGTCTACGGCGGCGGCAGCATCGGGCTGATGGGCTTGATCGCGCGTACGGTTCTCGACGGCGGCCGCCGT
GTCGTCGGGGTGATTCCTAGAGCTCTCATGGCTGTCGAGATATCAGGTGAGAGTGTGGGAGAAGTAATAGTTGT
ACAGGACATGCATGAGCGGAAAGCGGAGATGGCTCGGCGATCCAAAGCGTTCATTGCTCTTCCTGGGGGCTATG
GAACAATGGAGGAGCTGTTAGAGATGATAACATGGTGCCAACTTGGAATTCATGACAAGCCAGTTGGATTGCTA
AATGTTGACGGTTACTACGATCCACTGCTCGCGTTGTTCGACAAAGGCGAGGCGGAGGGCTTCATCAACTCTGA
TTGTAGACAAATATTTGTATCTGCACCAACTGCAAGTGAATTGCTGACAAAGATGGAGCAATACACTCGGTTGC
ACCAGGAGGTGGCCCCTGCAACAAGCTGGGAGATCTCAGAGCTTGGCTATGGAAGAACACCGGGCGCTGATCAA
TCCTAG
(SEQ ID NO: 4)
MGDGAEAAGATAASRFGTICVFCGSNAGRRRVFGDAALDLGHELVRRGVDLVYGGGSIGLMGLIARTVLDGGRR
VVGVIPRALMAVEISGESVGEVIVVQDMHERKAEMARRSKAFIALPGGYGTMEELLEMITWCQLGIHDKPVGLL
NVDGYYDPLLALFDKGEAEGFINSDCRQIFVSAPTASELLTKMEQYTRLHQEVAPATSWEISELGYGRTPGADQ
S

Homologs (e.g., >90% query cover and >80% sequence identity) of LOG1 in other species also include NP_001148565 in Zea mays (corn), XP_044349527 in Triticum aestivum (wheat), XP_044976839 in Hordeum vulgare (barley) and XP_002458381 in Sorghum bicolor (sorghum). The sequences are:

Zea mays (NP_001148565)
(SEQ ID NO: 5)
ATGGGCGATTCCGCGGCGGGCGCGCCGGAGCCGAGCAGGTTCGGCAGGATCTGCGTCTTCTGCGGCAGCAACCC
CGGCAATCGCGCCGTCTACGGGGACGCAGCGCTCGACCTCGGCAAAGAGCTGGTGGCGAAGGGGATCGATTTGG
TCTACGGCGGCGGGAGCGTCGGGCTCATGGGCCTGATCGCGCAGACGGTCCTTGGTGGCGGCTGCAGTGTCCTC
GGAGTGATTCCGAGAGCACTCATGCCGCTTGAGATATCTGGTGCTAGCGTTGGAGAAGTTAAGGTTGTCTCCGA
CATGCACGAGAGGAAAGCTGAGATGGCACGGCAAGCTGATGCCTTCATAGCTCTTCCTGGAGGGTACGGAACAA
TGGAAGAACTGTTAGAGATGATAACGTGGTCACAGCTCGGAATTCATGACAAACCAGTCGGTTTGCTGAACGTT
GATGGCTACTACGACCCGCTGCTCATGCTGTTTGATAGAGGGGCAACGGAAGGCTTTATCAAGCTAGATTGCAG
AGATATAATTGTTTCAGCGCCAACTGCCCATGAGTTACTGAAGAAGATGGAGCACTACACTCGGTCGCACCAGG
AGGTCGCCCCACGGACGAGCTGGGAGATGTCAGAGCTGGGCTATGGAAAAGCGTCGGAGTCATAG
(SEQ ID NO: 6)
MGDSAAGAPEPSRFGRICVFCGSNPGNRAVYGDAALDLGKELVAKGIDLVYGGGSVGLMGLIAQTVLGGGCSVL
GVIPRALMPLEISGASVGEVKVVSDMHERKAEMARQADAFIALPGGYGTMEELLEMITWSQLGIHDKPVGLLNV
DGYYDPLLMLFDRGATEGFIKLDCRDIIVSAPTAHELLKKMEHYTRSHQEVAPRTSWEMSELGYGKASES
Triticum aestivum (XP_044349527)
(SEQ ID NO: 7)
ATGCCGCCTTCATTTGAAGCTTCTCCCCCAGATACTCTACGAGATAACACACGGCATCGGGTAGAAGAACGAAC
CGAACCCAAACACCTCTCCTCCACGACGACTCTTAACCCCATCCTCCCGCCCCGCACCCGAGAGGAAACAAGCC
AGGCCATGGGCGACACCGCCGCGCCCGCGGCCGCGCCGCCGAGGAAGTTCGGCAGGATCTGCGTCTTCTGCGGC
AGCAACTCCGGCAATCGCGCGGTGTTCGGCGACACGGCGCTCGAGCTCGGCCAGGGGCTGGTGACGAGGGGGGT
TGATCTGGTCTATGGCGGGGGCAGCATCGGGTTGATGGGCCTGATCGCGCAGACGGTTCTGGATGGCGGCTGCC
GTGTCCTCGGGGTGATTCCCAGAGCACTCATGCCCCTTGAGATCTCTGGTGCAAGTGTTGGAGAAGTAAAGATA
GTCTCTGACATGCATGAGAGGAAAGCTGAGATGGCGCGACAAGCTGATGCATTCATTGCTCTTCCGGGAGGGTA
TGGAACAATGGAGGAGTTGTTAGAGATGATCACATGGTCGCAGCTCGGAATCCATGACAAACCAGTTGGCTTGC
TAAACGTCGACGGGTACTATGATCCGTTACTTGCGCTGTTCGACAAGGGCGCGGCAGAAGGTTTTATTAAGGCC
GATTGCAGGCAGATAATCGTGTCGGCGCCAACTGCCCACGAACTGCTGACGAAAATGGAGCAATACACCCGTTC
ACACCGGGAGGTGGCGTCGCGGACGAGCTGGGAGATGACCGAGCTGGGCTACGGGAAAGCTGCACCAGAGCCGG
AGGAGGAGGCGTCGTGCTGA
(SEQ ID NO: 8)
MPPSFEASPPDTLRDNTRHRVEERTEPKHLSSTTTLNPILPPRTREETSQAMGDTAAPAAAPPRKFGRICVFCG
SNSGNRAVFGDTALELGQGLVTRGVDLVYGGGSIGLMGLIAQTVLDGGCRVLGVIPRALMPLEISGASVGEVKI
VSDMHERKAEMARQADAFIALPGGYGTMEELLEMITWSQLGIHDKPVGLLNVDGYYDPLLALFDKGAAEGFIKA
DCRQIIVSAPTAHELLTKMEQYTRSHREVASRTSWEMTELGYGKAAPEPEEEASC
Hordeum vulgare (XP_044976839)
(SEQ ID NO: 9)
ATGGGCGACACCACCGCGCCCTCGCCGCCGAGGAGGTTCGGCAGGATCTGCGTCTTCTGCGGCAGCAACTCCGG
CAACCGCGCCGTGTTCGGCGACGCCGCGCTCGAGCTCGGCCAGGGCCTGGTGACGAGGGGGGTCGATCTGGTCT
ACGGCGGCGGCAGCATCGGGCTGATGGGCCTGATCGCGCAGACGGTTCTCGACGGCGGCTGCCGCGTCCTCGGG
GTGATTCCAAGAGCACTCATGCCCCTCGAGATATCCGGTGCAAGTGTTGGAGAAGTAAAGATTGTCTCCGACAT
GCATGAGAGGAAAGCTGAGATGGCGCGACAAGCCGATGCATTCATTGCTCTTCCGGGTGGGTATGGAACAATGG
AAGAGCTGTTAGAGATGATCACTTGGTCGCAGCTTGGAATCCATGACAAACCGGTCGGGTTGCTAAACGTCGAT
GGGTACTATGATCCGTTACTCGCGCTGTTCGACAAGGGCGCGGCGGAAGGGTTTATTAAGGCCGATTGCAGGCA
GATAATCGTGTCGGCACCAACTGCCCACGAACTGCTGACAAAAATGGAGCAATACACCCGTTCACACCGGGAGG
TGGCCTCGCGGACGAGCTGGGAGATGACCGAGATGGGCTACGGGAAAGCACCGGAGCCGGAGGAGGAGGCGGCG
GCATCGTAG
(SEQ ID NO: 10)
MGDTTAPSPPRRFGRICVFCGSNSGNRAVFGDAALELGQGLVTRGVDLVYGGGSIGLMGLIAQTVLDGGCRVLG
VIPRALMPLEISGASVGEVKIVSDMHERKAEMARQADAFIALPGGYGTMEELLEMITWSQLGIHDKPVGLLNVD
GYYDPLLALFDKGAAEGFIKADCRQIIVSAPTAHELLTKMEQYTRSHREVASRTSWEMTEMGYGKAPEPEEEAA
AS
Sorghum bicolor (XP_002458381)
(SEQ ID NO: 11)
ATGTGGGCTCCTGTGGTGCAATCTGGTGCGGCACTGGAGTTGACCCGGCAAATTGATTACGCGATTATTAAACA
GCACGATCGCGTCATGAGAGAAGCCCTGATGCCGCCTTCATTTGAAGCTCCCCCGTCCCGTCCCCTCTCCTCGA
AGGCAAAGAAGCCAAAGCCGAACACCTCACTTTATCCGCCGTCCCGCCGCCACTGCCCGCTCCGCTCCTCCGCC
ATGGGCGACACCGTAGCGGACGCGCCGGAGCCGAGCAGGTTCGGCAGGATCTGCGTCTTCTGCGGCAGCAACCC
CGGCAATCGCGCCGTCTACGGGGACGCCGCGCTCGACCTCGGCAAAGAGCTGGTGGCGAGGGGGATCGATTTGG
TCTACGGCGGCGGCAGCGTCGGGCTCATGGGCCTGATCGCGCAGACGGTTCTTGACGGCGGCTGCAGTGTCCTA
GGGGTGATTCCAAGAGCACTCATGCCGCTTGAGATATCTGGTGCTAGCGTTGGAGAAGTAAAGGTTGTCTCCGA
TATGCACGAAAGAAAAGCTGAGATGGCGCGACAAGCTGATGCCTTCATTGCTCTTCCTGGAGGGTATGGAACAA
TGGAAGAGCTGTTAGAGATGATAACATGGTCACAACTAGGAATTCATGATAAACCAGTTGGATTACTAAATGTT
GATGGTTACTACGACCCGTTGCTCATGCTATTTGACAAAGGGGCGACGGAAGGTTTTATTAAGCTAGATTGCAG
AGATATAATTGTTTCAGCACCAACTGCCCATGAATTGCTGGAGAAAATGGAGCACTACACTCGGTCACACCAGG
AGGTTGCCCCACGGACGAGCTGGGAGATGTCAGAGCTGGGCTATGGAAAAGCACCGGAGTCATAG
(SEQ ID NO: 12)
MWAPVVQSGAALELTRQIDYAIIKQHDRVMREALMPPSFEAPPSRPLSSKAKKPKPNTSLYPPSRRHCPLRSSA
MGDTVADAPEPSRFGRICVFCGSNPGNRAVYGDAALDLGKELVARGIDLVYGGGSVGLMGLIAQTVLDGGCSVL
GVIPRALMPLEISGASVGEVKVVSDMHERKAEMARQADAFIALPGGYGTMEELLEMITWSQLGIHDKPVGLLNV
DGYYDPLLMLFDKGATEGFIKLDCRDIIVSAPTAHELLEKMEHYTRSHQEVAPRTSWEMSELGYGKAPES

In accordance with the present invention, there may be employed conventional molecular biology, microbiology, biochemical, and recombinant DNA techniques within the 50 skill of the art. Such techniques are explained fully in the literature. The invention will be further described in the following examples, which do not limit the scope of the methods and compositions of matter described in the claims.

### EXAMPLES

### Example 1—Plant Material, HNT Treatment and GWAS

Rice Diversity Panel 1 (RDP1) consisting of accessions from different sub-populations was screened for natural variation in response to HNT stress during grain development. Rice seeds, sterilized with bleach for 40 minutes and soaked in water overnight were germinated on half strength Murashige and Skoog (MS) media for 2 d in dark, followed by 1 d growth in light. Six seedlings per accession were transplanted in 4-inch square pots that contained natural soil mix were grown under a controlled greenhouse diurnal setting with temperature 28/25±2° C., light/dark 16/8 h and relative humidity of 55-60%. When primary panicle reached 50% flowering, half of the plants from each accession were moved to HNT (30±1° C.: 8 h 28±1° C.) greenhouse. Mature dehulled grains from primary panicles were used for SGW analysis. To obtain dehulled fully developed grain weight was divided by number of grains. SGW data was further analyzed to obtain adjusted means for each accession across the replications using the following statistical model:

$${y}_{ik} = + {g}_{i} + {r}_{k} + {ε}_{ik}$$

where yᵢₖ refers to the performance of the ith accession in the kth replication, p is the intercept, gᵢ is the effect of the ith accession, rᵢ is the effect of kth replication, and 4 is the residual error associated with the observation yᵢₖ. All analyses was performed in the R environment (R Core Team, 2019). Further, the adjusted means of each accession were used for GWAS.

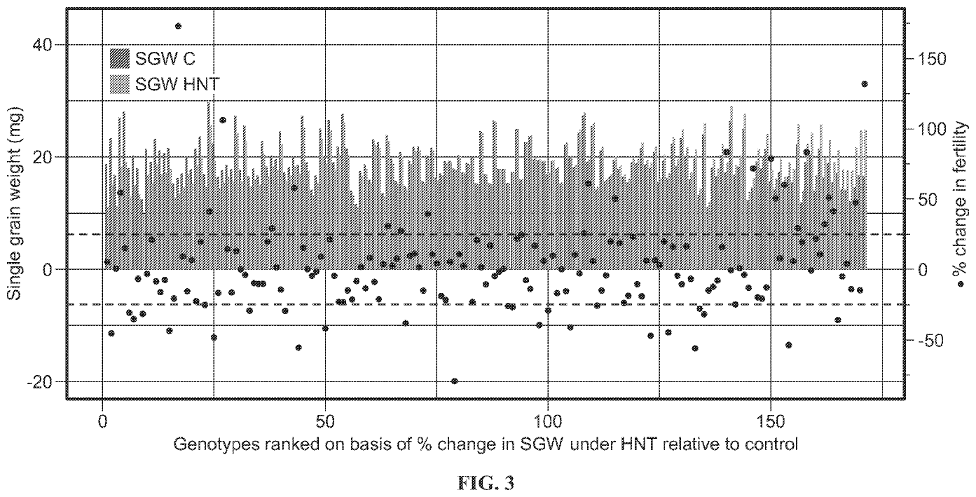

For GWAS analysis, a high-density rice array (HDRA) of a 700k single nucleotide polymorphism (SNP) marker dataset was used (McCouch et al., 2016, Nat. Commun., 7:10532). After filtering for the missing data (<20%) and minor allele frequency (<5%), 411 066 SNPs were retained for GWAS. Before GWAS, principle component analysis (PCA) was performed (Zheng et al., 2012, Sci. Rep., 2:888) to assess the population structure of the rice accessions (FIG. 3). Next, GWAS analysis was carried out in the R package, rrblup (Endelman, 2011, BMC Genomics, 12:407) using the following single marker linear mixed model:

$$y = 1 μ + X β + sa + Zg + ε$$

where, y is a vector of observations, μ is the overall mean, X is the design matrix for fixed effects, β is a vector of principle components accounting for population structure, s is a vector reflecting the number of alleles (0, 2) of each genotype at particular SNP locus, α is the effect of the SNP, Z is the design matrix for random effects, g is the vector of random effects accounting for relatedness and g˜N(0,Gσ_{g}²); G is the genomic relationship matrix of the genotypes, is the genetic variance, and c is the vector of residuals. The outputs generated from GWAS analysis were used to plot the Q-Q plots and Manhattan plots using the qqman package in R (Turner, 2014). The suggested threshold level of P<3.3×10⁻⁶ or −log 10(P)>5.4 was used to declare the genome-wide significance of SNP markers (Bai et al., 2016). Additionally, R2-values representing phenotypic variance contribution of each marker (or SNP) to the total variance were calculated using the bglr package (Pérez & De Los Campos, 2014, Genetics, 198(2):483-95). Narrow-sense heritability (h2) of the lead SNP with or without accounting for linkage disequilibrium (LD) was estimated by jointly fitting the lead SNP along with all the other SNPs or fitting the lead SNP alone via a genomic restricted maximum likelihood method (Yang et al., 2017) using the R package sommer (Covarrubias-Pazaran, 2016, PLoS One, 11(6):e0156744)

$${h}^{2} = \frac{{σ}_{g}^{2}}{{σ}_{g}^{2} + {σ}_{e}^{2}} ,$$

where σ_{g}² is the genetic variance and σ_{g}² is the residual variance.

### Example 2—Generation of LOG1 Mutants

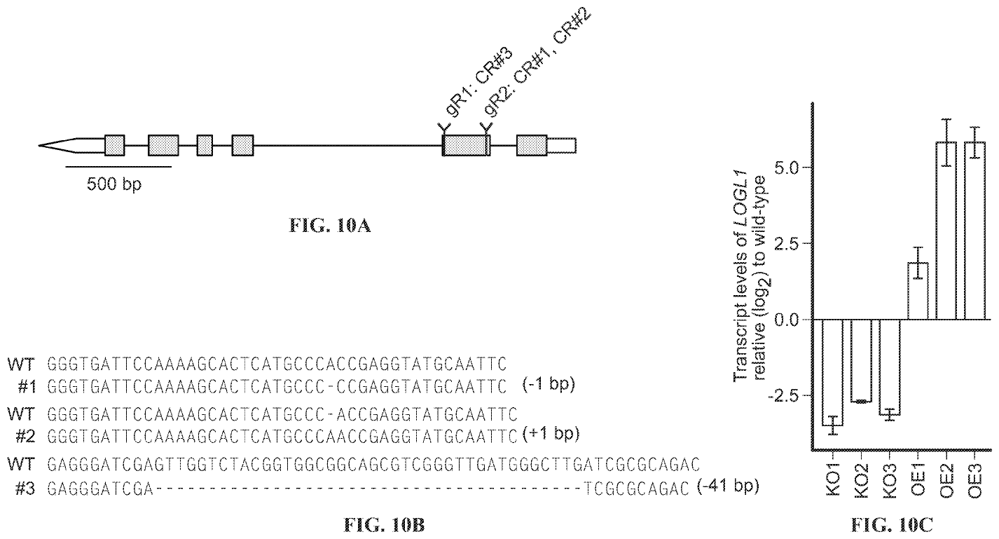

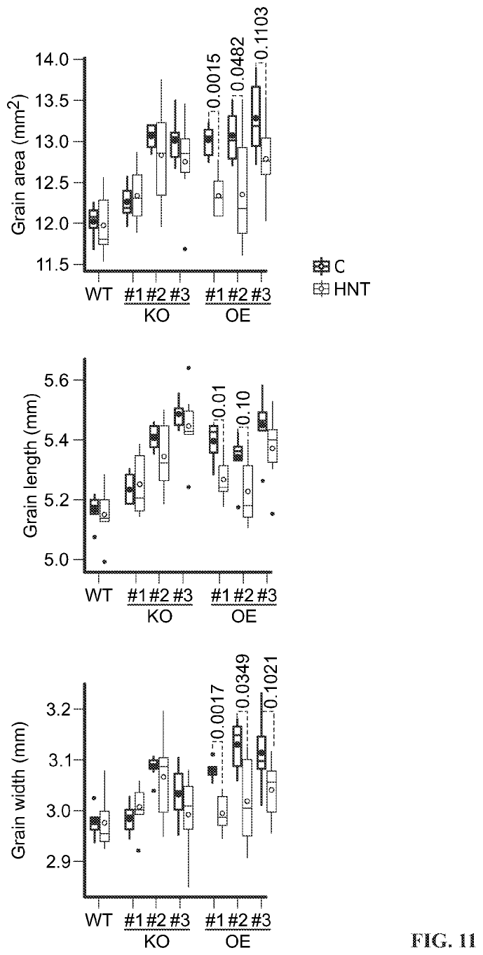

For CRISPR-Cas9 based knockout mutants, single-guide RNA (sgRNA) targeting 5′ end of the gene was designed using CRISPR-P 2.0 (crispr.hzau.edu.cn/CRISPR/on the World Wide Web) (Lei et al., 2014, Mol. Plant, 7:1494-96) (FIG. 10). Destination constructs were generated following a modified gateway cloning method described in Lowder et al. (2015, Plant Physiol., 169(2):971-85). Two single-guide sequences (sg1 and sg2) were designed to target two different sites. For this, two single-guide sequences (sg1 and sg2) cloned separately in pYPQ141C (using Esp3I/BsmBI site) was recombined in separate reactions with pANIC6B and pYPQ167 (Cas9) using LR-clonase (Table 1).

TABLE 1
Primers

| Purpose | | Genotypes | Gene | Primer Sequence (5′-3′) | SEQ ID |
|---|---|---|---|---|---|
| CRISPR CAS9-based | | KO#3 | LOG1 | gtgtGAGAGGGATCGAGTTGGTCTA | 13 |
| knock-out (KO) | | (sg1) | | aaacTAGACCAACTCGATCCCTCTC | 14 |
| | | KOs #1 | LOG1 | gtgtGAAAAGCACTCATGCCCACCG | 15 |
| | | and #2 | | aaacCGGTGGGCATGAGTGCTTTTC | 16 |
| | | (sg2) | | | |
| Over-expression (OE) | | OE #1, #2, | LOG1 | CACCATGGGCGACAACAGCGCC | 17 |
| | | #3 | | CGATTCCTCTGGTGTCTTTCCATAACC | 18 |
| KO | KO | KO#3 | LOG1 | CACCATGGGCGACAACAGCGCC | 19 |
| screening | screening: | (sg1) | | TACGTGATAGACCCTTGTGCC | 20 |
| | Primers of | KOs # | | CACAGAAAAGAGAAGCTGCCAA | 21 |
| | PCR | and | | CGCCATCAAGAACCGTCTGC | 22 |
| | amplification | #2 (sg2) | | | |
| | of target | | | | |
| | region | | | | |
| | Primers for | KO#3 | LOG1 | CACCATGGGCGACAACAGCGCC | 23 |
| | amplicon | (sg1) | | | |
| | sequencing | KOs # and | LOG1 | CGGGTTGATGGGCTTGATCG | 24 |
| | | #2 (sg2) | | | |
| Quantitative RT-PCR | | | LOG1 | CCGTTGCTTGCGCTATTTGA | 25 |
| | | | | ATCTCCCAGCTTGTACGTGG | 26 |
| | | | Reference | TCAACGTGGAGAAGCAGATG | 27 |
| | | | (Os03g63430) | ATGGCAGTGATAGCCTGCTT | 28 |

Agrobacterium tumefaciens strain EHA105 carrying the final CRISPR-Cas9 constructs (independent for sg1 and sg1) were used for rice callus transformation (Cheng et al., 1998, in Rice Transformation by Agrobacterium Infection, Humana Press; Chen et al., 2016, Plant Physiol., 171:606-22). Ti plants lacking Cas9 (confirmed using 03-glucuronidase assay) were screened for the presence of a mutation using Sanger sequencing. Finally, 3 KO lines (1 from s1 and 2 from sg2) were selected for downstream experiments. For overexpression lines, rice LOG1 coding region amplified from kitakee (cv) cDNA using specific primers was cloned int pENTRID-TOPO (Invitrogen). The entry construct was recombined with destination vector pANIC 6B with 35S promoter (35s::LOG1). The final destination construct was used to transform rice calli. Homozygous knockouts and overexpression plants from T3 or later generations were used for phenotypic evaluation. The expression of LOG1 in KO and OE lines were confirmed using RT-PCR in developing seeds.

### Example 3—Genomic DNA and RNA Extraction, RT-qPCR Assay

To screen for mutations in the knockouts, genomic DNA was isolated from seedling leaves. The primers flanking sgRNA site were used to amplify targeted region using Phusion reaction (Thermo Scientific). The resulting amplicon was genotyped using Sanger sequencing from eurofins. The resulting sequencing reads were aligned with wild-type sequence to decipher the mutations. RNA extraction and quantitative reverse transcription polymerase chain reaction (RT-qPCR) and analysis were performed as described previously. Briefly, RNA extraction were performed using Qiagen kit with addition of DNAase treatment. For cDNA synthesis, half microgram of RNA was used in 10 μl reverse transcription reaction using BioRad iscript. RT-qPCR was conducted using 2 μl of diluted cDNA (1:10) in 10 μl Roche Syber Green reaction.

### Example 4—Results

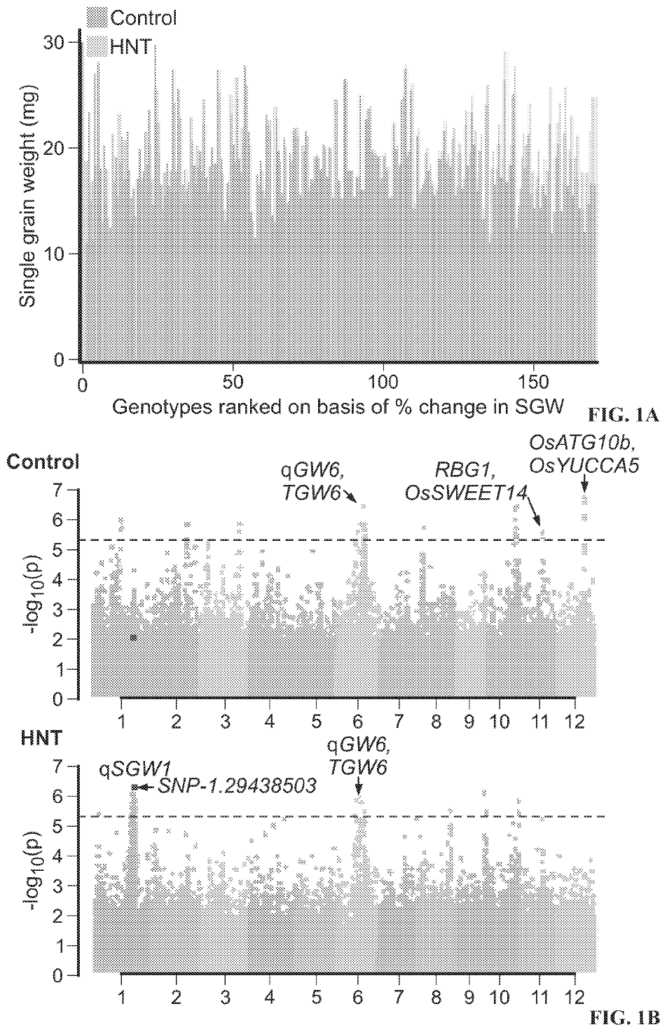

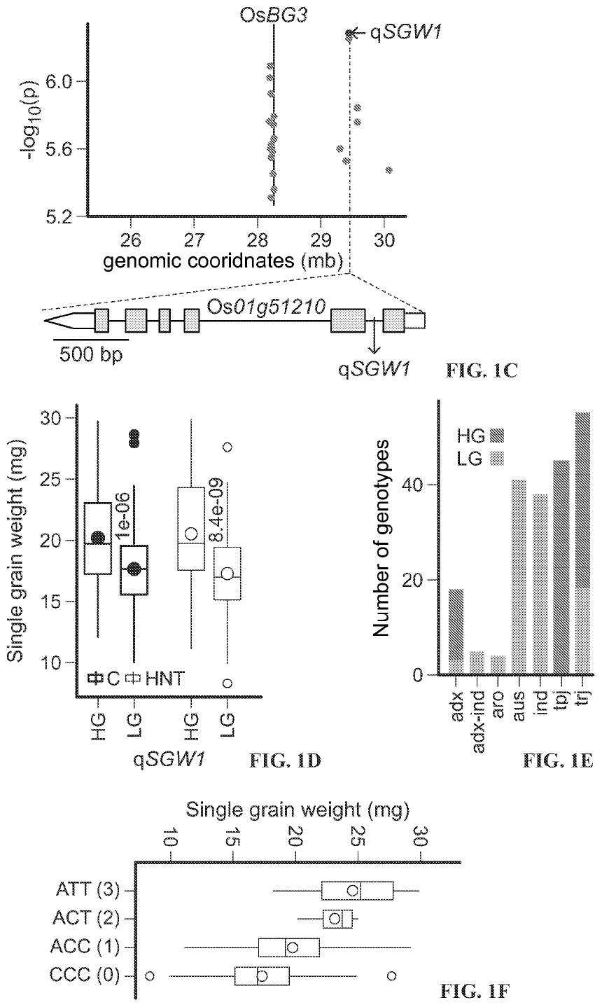

A terminal HNT stress treatment (give temps here) was imposed during grain development on a diverse set of 221 rice accessions from Rice Diversity Panel (RDP1) (Eizenga et al., 2014, J. Plant Regist., 8:109-16; Ali et al., 2011, Crop Sci., 51:2021-35; Huang et al., 2010, Nat. Genet., 42:961-7). The single grain weight (SGW) of individually marked grain from mature primary panicles was measured as the main yield component for HNT tolerance in this study (Impa et al., 2021, Plant. Cell Environ., 44:2049-65; Li et al., 2021, Crop, 9:577-89; Zhai et al., 2020, Front. Plant Sci., 11:933). A significant variation in SGW across the accessions under both control (mean=19 mg, range=10-29.7 mg) and HNT (mean=18.8 mg, range=8.31-29.1 mg) treatments was observed (FIG. 1A). The percentage change in SGW of HNT-treated seeds was examined relative to control seeds and 66 accessions were found with more than a 5% decrease (referred to as sensitive) and 48 accessions with more than a 5% increase (tolerant) in SGW under HNT. This suggested that rice germplasm has considerable variation for HNT stress response at single grain level. Given the plastic relationship between grain number and weight in rice (REF 15-19), we asked if higher SGW for tolerant accessions under HNT stress was due to reduced fertility (hence, lower grain number). Sensitive and tolerant accessions were selected as defined by the 5% SGW threshold and the panicle level fertility of these accessions was examined. No clear association (Fisher's Exact Test p-value=0.256, Table 2, FIG. 3) exists between fertility and SGW response to HNT stress. Therefore, higher SGW of tolerant accessions is not due to reduced fertility. Additionally, the data suggest that 28 of the 48 tolerant accessions have lower sensitivity for fertility under HNT stress (Table 2). Briefly, the study identified several HNT-tolerant lines that are able to either maintain or have improved SGW and fertility under HNT.

TABLE 2
Relationship between HNT response of single grain weight (SGW) and panicle fertility

| | | Fertility | |
|---|---|---|---|
| | | Sensitive | Tolerant |
| SGW | | more than 5% reduction | % change >−5% |
| | % change in SGW | 32 | 27 |
| Sensitive | more than 5% reduction | 27 | 39 |
| Moderately tolerant | increase or decrease is <5% | 20 | 28 |
| Tolerant | more than 5% increase | | |

Genome-wide association analysis on the SGW trait was performed under control and HNT stress independently and 33 and 44 distinct loci for control and HNT stress were identified, respectively, from a total of 67 significant SNPs (FIG. 1B-1C; Table 3). Notably, eight loci were specific to HNT stress (Chr1: qtl-1.2, 1.3, 1.4, 1.5, 1.6, 1.7; Chr8: qtl-8.2; Chr:qtl-9.1), and six were identified in control (Chr1: qtl-1.1; Chr2: qtl-2.1; Chr3: qtl-3.1; Chr8: qtl-8.1; Chr11: qtl-11.1; Chr12: qtl-12.1) treatment only (Table 3). Two significant peaks on chromosome 6 spanning between 15-20.5 Mb for control and from 13-20 Mb for HNT stress treatment co-localized with a previously identified QTL for grain weight under optimal temperature on chr 6, qGW6 (Ngu et al., 2014, Genet. Mol. Res., 13:9477-88). Within this peak (referred as qSGW6), SNP-6.17579336, SNP-6.20133990, SNP-6.20143494, and SNP-6.20150777 showed significant association with SGW under both control and HNT conditions. The phenotypic variation explained by these SNPs ranged between 8-9% under both control and HNT. The SNP-6.20150777 is located ˜5 Mb upstream of THOUSAND-GRAIN WEIGHT 6 (TGW6), which regulates rice grain weight by controlling IAA supply in developing endosperm (Ishimaru et al., 2013, Nat. Genet., 45:707-11). The most significant SNP (SNP-12.19515276, −log₁₀(p)=6.74, R2=0.11) under control conditions was within an association peak (span 19.3-19.5 Mb) detected on chr 12. This region was scanned for the presence of previously characterized genes and it was found that OsATG10b (Os12g32210) is located within this association peak and is 100 kb upstream of lead SNP (Shin et al., 2009, Mol. Cells, 27:67-74). Further, another gene involved in IAA synthesis, OsYUCCA5, is localized within 250 kb downstream of this lead SNP (Li et al., 2021, J. Integr. Plant Biol., 63:1521-37). Most of the other genes in this region encode for either expressed protein or transposon category. However, causal gene underlying this locus that controls natural variation in SGW still remain unknown. A pair of control temperature-specific SNPs were detected on chr 11. These two SNPs are upstream (SNP-11.17221237, position: 17707382) and downstream (SNP-11.17705601, position: 18191744) of Rice Big Grain 1 (RBG1). RBG1 promotes cell division that leads to increased organ size and hence, yield. RBG1 also upregulates the expression of heat shock proteins, leading to improved abiotic stress tolerance in RBG1-overexpression plants (Lo et al., 2020, Plant Biotechnol. J., 18:1969-83). It was found that OsSWEET14 is located 20 kb downstream of SNP-11.17705601. OsSWEET14 encodes for a sucrose efflux protein that regulates supply to developing endosperm during grain filling stage (Fei et al., 2021, Plant Sci., 306). Collectively, co-localization of several significant SNPs with previously known yield regulators validated our GWAS results.

TABLE 3
GWAS-based significant SNPs associated with single grain weight under control and
HNT. Genes in vicinity of significant SNPs (location based on MSUv7.0) are listed

| Treatment | Chromosome | Walia_QTL | SNP_ID | SNP_Position | log10P(−ve) | Gene_start | Gene_Stop | MSU_ID |
|---|---|---|---|---|---|---|---|---|
| control | 1 | qtl1.1 | SNP-1.20013921. | 20014967 | 5.96 | 20001505 | 20008180 | LOC_Os01g36170 |
| | | | | | | 20010148 | 20013007 | LOC_Os01g36180 |
| | | | | | | 20023159 | 20023440 | LOC_Os01g36200 |
| | | | | | | 20033503 | 20035006 | LOC_Os01g36220 |
| | | | SNP-1.20302781. | 20303827 | 5.68 | 20284447 | 20287465 | LOC_Os01g36550 |
| | | | | | | 20287702 | 20290661 | LOC_Os01g36560 |
| | | | | | | 20292590 | 20292886 | LOC_Os01g36570 |
| | | | | | | 20299522 | 20302467 | LOC_Os01g36580 |
| | | | | | | 20304884 | 20310385 | LOC_Os01g36590 |
| | | | | | | 20312945 | 20314180 | LOC_Os01g36600 |
| | | | | | | 20314316 | 20316106 | LOC_Os01g36610 |
| | | | | | | 20317862 | 20319378 | LOC_Os01g36620 |
| | | | | | | 20322698 | 20326376 | LOC_Os01g36630 |
| hnt | 1 | qtl1.2 | SNP-1.2238756. | 2239757 | 5.39 | 2233636 | 2234508 | LOC_Os01g04850 |
| | | | | | | 2215586 | 2222837 | LOC_Os01g04814 |
| | | | | | | 2224683 | 2225060 | LOC_Os01g04830 |
| | | | | | | 2226000 | 2229526 | LOC_Os01g04840 |
| | | | | | | 2245732 | 2250576 | LOC_Os01g04860 |
| | | | | | | 2250820 | 2255073 | LOC_Os01g04870 |
| hnt | 1 | qtl1.3 | SNP-1.28185856. | 28186901 | 5.76 | 28161732 | 28166115 | LOC_Os01g49010 |
| | | | SNP-1.28193184. | 28194229 | 6.09 | 28166404 | 28167210 | LOC_Os01g49020 |
| | | | SNP-1.28199651. | 28200696 | 5.60 | 28167063 | 28172563 | LOC_Os01g49024 |
| | | | SNP-1.28200702. | 28201747 | 5.93 | 28173827 | 28174830 | LOC_Os01g49026 |
| | | | SNP-1.28201868. | 28202913 | 5.31 | 28176743 | 28177756 | LOC_Os01g49030 |
| | | | SNP-1.28209666. | 28210711 | 5.75 | 28180868 | 28181677 | LOC_Os01g49050 |
| | | | SNP-1.28209817. | 28210862 | 5.55 | 28187853 | 28193358 | LOC_Os01g49060 |
| | | | SNP-1.28213569. | 28214614 | 5.63 | 28193378 | 28196715 | LOC_Os01g49065 |
| | | | SNP-1.28220835. | 28221880 | 5.58 | 28197980 | 28198294 | LOC_Os01g49070 |
| | | | SNP-1.28234464. | 28235509 | 5.75 | 28209112 | 28210954 | LOC_Os01g49080 |
| | | | SNP-1.28237920. | 28238965 | 5.65 | 28218457 | 28220215 | LOC_Os01g49090 |
| | | | SNP-1.28240500. | 28241545 | 5.45 | 28231417 | 28235420 | LOC_Os01g49110 |
| | | | SNP-1.28240716. | 28241761 | 5.66 | 28234702 | 28238634 | LOC_Os01g49120 |
| | | | SNP-1.28251560. | 28252605 | 5.36 | 28240069 | 28240557 | LOC_Os01g49130 |
| | | | SNP-1.28254946. | 28255991 | 5.79 | 28242146 | 28246259 | LOC_Os01g49150 |
| | | | | | | 28246607 | 28248021 | LOC_Os01g49154 |
| | | | | | | 28249920 | 28251516 | LOC_Os01g49160 |
| | | | | | | 28261415 | 28273067 | LOC_Os01g49180 |
| hnt | 1 | qtl1.4 | SNP-1.29290615. | 29291661 | 5.60 | 29271158 | 29273270 | LOC_Os01g50940 |
| | | | | | | 29277487 | 29277807 | LOC_Os01g50950 |
| | | | | | | 29280126 | 29282901 | LOC_Os01g50960 |
| | | | | | | 29285372 | 29288718 | LOC_Os01g50970 |
| | | | | | | 29290326 | 29295442 | LOC_Os01g50980 |
| | | | | | | 29298041 | 29299464 | LOC_Os01g50990 |
| | | | | | | 29301260 | 29302430 | LOC_Os01g51000 |
| | | | | | | 29306448 | 29312618 | LOC_Os01g51010 |
| | | | | | | 29309116 | 29312618 | LOC_Os01g51010 |
| hnt | 1 | qtl1.5/ qSGW1.1 | SNP-1.29392387. | 29393433 | 5.53 | 29369748 | 29376123 | LOC_Os01g51130 |
| | | | SNP-1.29427477. | 29428523 | 6.26 | 29382534 | 29385653 | LOC_Os01g51140 |
| | | | SNP-1.29438503. | 29439549 | 6.28 | 29405623 | 29411374 | LOC_Os01g51154 |
| | | | | | | 29412196 | 29413972 | LOC_Os01g51170 |
| | | | | | | 29416278 | 29423224 | LOC_Os01g51180 |
| | | | | | | 29423421 | 29428115 | LOC_Os01g51190 |
| | | | | | | 29431601 | 29436466 | LOC_Os01g51200 |
| | | | | | | 29437276 | 29439882 | LOC_Os01g51210 |
| | | | | | | 29442650 | 29447144 | LOC_Os01g51220 |
| | | | | | | 29447990 | 29450454 | LOC_Os01g51230 |
| | | | | | | 29454654 | 29455461 | LOC_Os01g51240 |
| | | | | | | 29458644 | 29463035 | LOC_Os01g51250 |
| hnt | 1 | qtl1.6 | SNP-1.29565777. | 29566823 | 5.85 | 29544661 | 29548524 | LOC_Os01g51390 |
| | | | SNP-1.29568499. | 29569545 | 5.76 | 29551666 | 29554002 | LOC_Os01g51400 |
| | | | | | | 29560350 | 29567065 | LOC_Os01g51410 |
| | | | | | | 29568547 | 29571484 | LOC_Os01g51420 |
| | | | | | | 29572653 | 29575706 | LOC_Os01g51430 |
| | | | | | | 29578534 | 29578983 | LOC_Os01g51440 |
| | | | | | | 29580715 | 29583174 | LOC_Os01g51450 |
| | | | | | | 29583876 | 29584603 | LOC_Os01g51460 |
| | | | | | | 29585902 | 29586405 | LOC_Os01g51470 |
| | | | | | | 29587120 | 29587624 | LOC_Os01g51480 |
| | | | | | | 29589249 | 29590343 | LOC_Os01g51490 |
| hnt | 1 | qtl1.7 | SNP-1.30064707. | 30065753 | 5.48 | 30048291 | 30049986 | LOC_Os01g52270 |
| | | | | | | 30055567 | 30057039 | LOC_Os01g52280 |
| | | | | | | 30058008 | 30060963 | LOC_Os01g52290 |
| | | | | | | 30062716 | 30066346 | LOC_Os01g52304 |
| | | | | | | 30068605 | 30070131 | LOC_Os01g52320 |
| | | | | | | 30075624 | 30077126 | LOC_Os01g52330 |
| | | | | | | 30079649 | 30081500 | LOC_Os01g52340 |
| control | 2 | qtl2.1 | SNP-2.25945650. | 25951520 | 5.32 | 25931511 | 25933904 | LOC_Os02g43060 |
| | | | | | | 25937172 | 25938413 | LOC_Os02g43070 |
| | | | | | | 25940341 | 25944110 | LOC_Os02g43080 |
| | | | | | | 25946390 | 25951088 | LOC_Os02g43090 |
| | | | | | | 25955402 | 25956356 | LOC_Os02g43100 |
| | | | | | | 25957160 | 25959377 | LOC_Os02g43110 |
| | | | | | | 25967752 | 25968734 | LOC_Os02g43120 |
| | | | SNP-2.26031313. | 26037183 | 5.80 | 26021498 | 26021959 | LOC_Os02g43160 |
| | | | | | | 26027785 | 26029493 | LOC_Os02g43170 |
| | | | | | | 26031508 | 26033900 | LOC_Os02g43180 |
| | | | | | | 26034320 | 26041422 | LOC_Os02g43194 |
| | | | | | | 26042360 | 26043139 | LOC_Os02g43210 |
| | | | | | | 26046025 | 26046681 | LOC_Os02g43220 |
| | | | | | | 26047569 | 26048033 | LOC_Os02g43230 |
| | | | | | | 26052820 | 26054216 | LOC_Os02g43250 |
| | | | | | | 26056918 | 26062077 | LOC_Os02g43260 |
| | | | SNP-2.26259529. | 26265399 | 5.86 | 26243664 | 26246240 | LOC_Os02g43480 |
| | | | | | | 26246799 | 26248165 | LOC_Os02g43490 |
| | | | | | | 26248624 | 26252008 | LOC_Os02g43500 |
| | | | | | | 26253018 | 26253744 | LOC_Os02g43510 |
| | | | | | | 26262647 | 26265824 | LOC_Os02g43519 |
| | | | | | | 26267434 | 26267727 | LOC_Os02g43530 |
| | | | | | | 26271679 | 26272657 | LOC_Os02g43540 |
| | | | | | | 26275370 | 26277684 | LOC_Os02g43550 |
| | | | | | | 26280253 | 26283914 | LOC_Os02g43560 |
| control | 3 | qtl3.1 | SNP-3.29098682. | 29105631 | 5.59 | 29088611 | 29093429 | LOC_Os03g50920 |
| | | | | | | 29098595 | 29105586 | LOC_Os03g50940 |
| | | | | | | 29108322 | 29113122 | LOC_Os03g50960 |
| | | | | | | 29112636 | 29121892 | LOC_Os03g50970 |
| | | | SNP-3.29413639. | 29420600 | 5.84 | 29399950 | 29405115 | LOC_Os03g51390 |
| | | | | | | 29409791 | 29414144 | LOC_Os03g51420 |
| | | | | | | 29395594 | 29403406 | LOC_Os03g51380 |
| | | | | | | 29419116 | 29427439 | LOC_Os03g51430 |
| | | | | | | 29424346 | 29432915 | LOC_Os03g51440 |
| control | 6 | qt6.1 | SNP-6.15005041. | 15006041 | 5.57 | 14984597 | 14986852 | LOC_Os06g25630 |
| | | | | | | 14987790 | 14988185 | LOC_Os06g25640 |
| | | | | | | 14988530 | 14990774 | LOC_Os06g25650 |
| | | | | | | 14991041 | 14994387 | LOC_Os06g25660 |
| | | | | | | 14995185 | 15004048 | LOC_Os06g25670 |
| | | | | | | 15006065 | 15008361 | LOC_Os06g25680 |
| | | | | | | 15010498 | 15012114 | LOC_Os06g25690 |
| | | | | | | 15013322 | 15014185 | LOC_Os06g25700 |
| | | | | | | 15015289 | 15018637 | LOC_Os06g25710 |
| | | | | | | 15020222 | 15021025 | LOC_Os06g25720 |
| control | | qt6.2 | SNP-6.17579336. | 17580334 | 5.82 | 17555785 | 17562493 | LOC_Os06g30390 |
| | | | | | | 17565595 | 17566008 | LOC_Os06g30400 |
| | | | | | | 17576997 | 17578925 | LOC_Os06g30420 |
| | | | | | | 17584536 | 17586350 | LOC_Os06g30430 |
| | | | | | | 17586899 | 17590682 | LOC_Os06g30440 |
| | | | | | | 17593381 | 17593980 | LOC_Os06g30450 |
| | | | | | | 17597786 | 17609675 | LOC_Os06g30460 |
| | | | SNP-6.19586948. | 19587946 | 6.45 | 19564209 | 19569249 | LOC_Os06g33610 |
| | | | | | | 19576632 | 19577366 | LOC_Os06g33620 |
| | | | | | | 19578859 | 19579158 | LOC_Os06g33630 |
| | | | | | | 19581998 | 19585408 | LOC_Os06g33640 |
| | | | | | | 19586425 | 19589623 | LOC_Os06g33650 |
| | | | | | | 19593746 | 19595295 | LOC_Os06g33660 |
| | | | | | | 19599277 | 19603396 | LOC_Os06g33680 |
| | | | | | | 19604055 | 19606116 | LOC_Os06g33690 |
| | | | SNP-6.20025944. | 20026942 | 5.30 | 20006378 | 20007736 | LOC_Os06g34370 |
| | | | | | | 20008346 | 20009575 | LOC_Os06g34380 |
| | | | | | | 20010528 | 20012036 | LOC_Os06g34390 |
| | | | | | | 20019172 | 20021317 | LOC_Os06g34400 |
| | | | | | | 20025001 | 20025327 | LOC_Os06g34410 |
| | | | | | | 20025972 | 20031155 | LOC_Os06g34420 |
| | | | | | | 20033279 | 20034343 | LOC_Os06g34430 |
| | | | | | | 20034666 | 20042684 | LOC_Os06g34440 |
| | | | SNP-6.20143494. | 20144492 | 5.67 | 20121525 | 20125289 | LOC_Os06g34600 |
| | | | | | | 20126199 | 20126870 | LOC_Os06g34610 |
| | | | | | | 20131644 | 20132168 | LOC_Os06g34620 |
| | | | | | | 20134621 | 20135274 | LOC_Os06g34630 |
| | | | | | | 20138220 | 20138690 | LOC_Os06g34640 |
| | | | | | | 20143793 | 20144666 | LOC_Os06g34650 |
| | | | | | | 20145118 | 20148099 | LOC_Os06g34660 |
| | | | | | | 20149011 | 20150251 | LOC_Os06g34670 |
| | | | | | | 20150646 | 20151752 | LOC_Os06g34680 |
| | | | | | | 20158641 | 20165624 | LOC_Os06g34690 |
| | | | SNP-6.20149468. | 20150466 | 5.36 | 20166745 | 20168754 | LOC_Os06g34700 |
| | | | SNP-6.20150777. | 20151775 | 5.83 | 20169366 | 20177261 | LOC_Os06g34710 |
| | | | SNP-6.20169389. | 20170387 | 5.65 | 20178120 | 20179043 | LOC_Os06g34730 |
| | | | SNP-6.20564952. | 20565950 | 5.46 | 20546705 | 20552296 | LOC_Os06g35220 |
| | | | | | | 20553372 | 20553865 | LOC_Os06g35230 |
| | | | | | | 20555172 | 20555504 | LOC_Os06g35240 |
| | | | | | | 20556063 | 20558644 | LOC_Os06g35250 |
| | | | | | | 20559047 | 20560934 | LOC_Os06g35260 |
| | | | | | | 20561504 | 20564441 | LOC_Os06g35270 |
| | | | | | | 20574703 | 20576020 | LOC_Os06g35280 |
| | | | | | | 20578770 | 20579267 | LOC_Os06g35290 |
| | | | | | | 20581980 | 20583058 | LOC_Os06g35300 |
| | | | | | | 20584398 | 20585649 | LOC_Os06g35310 |
| hnt | 6 | qt6.3 | SNP-6.13293299. | 13294299 | 5.31 | 13278774 | 13279302 | LOC_Os06g22800 |
| | | | | | | 13297087 | 13301279 | LOC_Os06g22810 |
| | | | | | | 13302546 | 13306574 | LOC_Os06g22820 |
| | | | | | | 14394105 | 14394455 | LOC_Os06g24550 |
| | | | | | | 14398932 | 14399252 | LOC_Os06g24560 |
| | | | SNP-6.14410668. | 14411668 | 5.86 | 14402929 | 14408551 | LOC_Os06g24570 |
| | | | | | | 14414517 | 14415628 | LOC_Os06g24580 |
| | | | | | | 14418161 | 14428791 | LOC_Os06g24594 |
| hnt | 6 | qt6.4 | SNP-6.17579336. | 17580334 | 5.76 | 17555785 | 17562493 | LOC_Os06g30390 |
| | | | | | | 17565595 | 17566008 | LOC_Os06g30400 |
| | | | | | | 17576997 | 17578925 | LOC_Os06g30420 |
| | | | | | | 17584536 | 17586350 | LOC_Os06g30430 |
| | | | | | | 17586899 | 17590682 | LOC_Os06g30440 |
| | | | | | | 17593381 | 17593980 | LOC_Os06g30450 |
| | | | | | | 17597786 | 17609675 | LOC_Os06g30460 |
| | | | SNP-6.20150777. | 20151775 | 5.46 | 20131644 | 20132168 | LOC_Os06g34620 |
| | | | | | | 20134621 | 20135274 | LOC_Os06g34630 |
| | | | | | | 20138220 | 20138690 | LOC_Os06g34640 |
| | | | | | | 20143793 | 20144666 | LOC_Os06g34650 |
| | | | | | | 20145118 | 20148099 | LOC_Os06g34660 |
| | | | | | | 20149011 | 20150251 | LOC_Os06g34670 |
| | | | | | | 20150646 | 20151752 | LOC_Os06g34680 |
| | | | | | | 20158641 | 20165624 | LOC_Os06g34690 |
| | | | | | | 20166745 | 20168754 | LOC_Os06g34700 |
| | | | | | | 20169366 | 20177261 | LOC_Os06g34710 |
| control | 8 | qtl8.1 | SNP-8.3194385. | 3195383 | 5.74 | 3171542 | 3175438 | LOC_Os08g05890 |
| | | | | | | 3175634 | 3176002 | LOC_Os08g05900 |
| | | | | | | 3182309 | 3190451 | LOC_Os08g05910 |
| | | | | | | 3192389 | 3196620 | LOC_Os08g05920 |
| | | | | | | 3199423 | 3205071 | LOC_Os08g05930 |
| hnt | 8 | qtl8.2 | SNP-8.23666465. | 23669180 | 5.50 | 23648009 | 23651153 | LOC_Os08g37390 |
| | | | | | | 23663492 | 23665549 | LOC_Os08g37400 |
| | | | | | | 23668965 | 23671157 | LOC_Os08g37410 |
| hnt | 9 | qtl9.1 | SNP-9.20497102. | 20497584 | 6.09 | 20506549 | 20508035 | LOC_Os09g35650 |
| | | | | | | 20509697 | 20513875 | LOC_Os09g35660 |
| | | | | | | 20485716 | 20486552 | LOC_Os09g35620 |
| | | | | | | 20494054 | 20496911 | LOC_Os09g35630 |
| | | | | | | 20500761 | 20501048 | LOC_Os09g35640 |
| | | | SNP-9.22196469. | 22196951 | 5.38 | 22173732 | 22178361 | LOC_Os09g38550 |
| | | | SNP-9.22217896. | 22218378 | 5.46 | 22181587 | 22183290 | LOC_Os09g38560 |
| | | | | | | 22188092 | 22191842 | LOC_Os09g38570 |
| | | | | | | 22192725 | 22197187 | LOC_Os09g38580 |
| | | | | | | 22199865 | 22200557 | LOC_Os09g38590 |
| | | | | | | 22202078 | 22204168 | LOC_Os09g38600 |
| | | | | | | 22207643 | 22208502 | LOC_Os09g38610 |
| | | | | | | 22212390 | 22218252 | LOC_Os09g38620 |
| | | | | | | 22220410 | 22221987 | LOC_Os09g38630 |
| | | | | | | 22223040 | 22224816 | LOC_Os09g38640 |
| | | | | | | 22225227 | 22226831 | LOC_Os09g38650 |
| | | | | | | 22230291 | 22232785 | LOC_Os09g38660 |
| | | | | | | 22233617 | 22235974 | LOC_Os09g38670 |
| | | | | | | 22237627 | 22238426 | LOC_Os09g38680 |
| control | 10 | qtl10.1 | SNP-10.19827101. | 19898602 | 5.47 | 19881378 | 19883255 | LOC_Os10g37160 |
| | | | SNP-10.19857169. | 19928670 | 5.52 | 19884257 | 19888276 | LOC_Os10g37170 |
| | | | | | | 19894708 | 19896859 | LOC_Os10g37180 |
| | | | | | | 19899083 | 19900928 | LOC_Os10g37190 |
| | | | | | | 19908434 | 19913891 | LOC_Os10g37210 |
| | | | | | | 19914170 | 19914844 | LOC_Os10g37220 |
| | | | | | | 19915687 | 19916952 | LOC_Os10g37230 |
| | | | | | | 19930005 | 19934782 | LOC_Os10g37240 |
| | | | SNP-10.20609128. | 20680650 | 6.31 | 20660761 | 20663311 | LOC_Os10g38820 |
| | | | | | | 20671799 | 20678392 | LOC_Os10g38834 |
| | | | | | | 20681136 | 20689084 | LOC_Os10g38850 |
| | | | | | | 20692550 | 20696256 | LOC_Os10g38860 |
| | | | SNP-10.20675661. | 20747183 | 6.00 | 20725051 | 20731000 | LOC_Os10g38900 |
| | | | | | | 20729297 | 20731000 | LOC_Os10g38910 |
| | | | | | | 20731150 | 20734054 | LOC_Os10g38920 |
| | | | | | | 20736983 | 20738313 | LOC_Os10g38930 |
| | | | | | | 20744421 | 20746446 | LOC_Os10g38940 |
| | | | | | | 20756089 | 20760021 | LOC_Os10g38950 |
| | | | | | | 20759602 | 20760894 | LOC_Os10g38954 |
| | | | | | | 20764158 | 20766182 | LOC_Os10g38960 |
| | | | SNP-10.20809470. | 20880992 | 5.69 | 20862952 | 20873797 | LOC_Os10g39130 |
| | | | | | | 20886352 | 20892217 | LOC_Os10g39140 |
| | | | | | | 20893498 | 20895220 | LOC_Os10g39150 |
| | | | | | | 20895706 | 20896832 | LOC_Os10g39160 |
| | | | | | | 20900132 | 20902342 | LOC_Os10g39170 |
| | | | SNP-10.20887390. | 20958912 | 5.39 | 20940762 | 20947963 | LOC_Os10g39230 |
| | | | | | | 20955031 | 20955862 | LOC_Os10g39250 |
| | | | | | | 20964775 | 20966829 | LOC_Os10g39280 |
| | | | | | | 20936559 | 20940489 | LOC_Os10g39220 |
| | | | | | | 20956882 | 20958393 | LOC_Os10g39260 |
| | | | | | | 20961133 | 20962326 | LOC_Os10g39270 |
| | | | | | | 20971236 | 20972702 | LOC_Os10g39300 |
| | | | SNP-10.21243375 | 21314898 | 5.48 | 21322788 | 21327370 | LOC_Os10g39850 |
| | | | | | | 21292884 | 21297500 | LOC_Os10g39780 |
| | | | | | | 21298411 | 21301728 | LOC_Os10g39790 |
| | | | | | | 21302009 | 21305661 | LOC_Os10g39800 |
| | | | | | | 21308453 | 21311416 | LOC_Os10g39810 |
| | | | | | | 21311579 | 21315431 | LOC_Os10g39820 |
| | | | | | | 21316350 | 21319353 | LOC_Os10g39830 |
| | | | | | | 21320189 | 21322235 | LOC_Os10g39840 |
| | | | | | | 21327674 | 21330652 | LOC_Os10g39860 |
| | | | | | | 21331492 | 21332655 | LOC_Os10g39870 |
| | | | | | | 21334654 | 21335827 | LOC_Os10g39880 |
| | | | SNP-10.21363510 | 21435034 | 6.42 | 21417293 | 21419897 | LOC_Os10g39980 |
| | | | | | | 21424365 | 21425634 | LOC_Os10g39990 |
| | | | | | | 21426957 | 21428453 | LOC_Os10g40000 |
| | | | | | | 21430780 | 21431821 | LOC_Os10g40010 |
| | | | | | | 21434984 | 21439378 | LOC_Os10g40020 |
| | | | | | | 21440267 | 21443607 | LOC_Os10g40030 |
| | | | | | | 21444257 | 21445487 | LOC_Os10g40040 |
| | | | | | | 21454034 | 21459779 | LOC_Os10g40060 |
| | | | SNP-10.21781643. | 21853168 | 5.34 | 21835930 | 21837651 | LOC_Os10g40710 |
| | | | | | | 21847804 | 21849726 | LOC_Os10g40720 |
| | | | | | | 21858750 | 21861307 | LOC_Os10g40730 |
| | | | | | | 21863008 | 21867667 | LOC_Os10g40740 |
| hnt | 10 | qtl10.2 | SNP-10.23114642. | 23186171 | 5.43 | 23165582 | 23167298 | LOC_Os10g42980 |
| | | | SNP-10.23134409. | 23205938 | 5.83 | 23169453 | 23175730 | LOC_Os10g42999 |
| | | | | | | 23179800 | 23181912 | LOC_Os10g43020 |
| | | | | | | 23185158 | 23185802 | LOC_Os10g43030 |
| | | | | | | 23186413 | 23189553 | LOC_Os10g43040 |
| | | | | | | 23190115 | 23192319 | LOC_Os10g43050 |
| | | | | | | 23192342 | 23195088 | LOC_Os10g43060 |
| | | | | | | 23195740 | 23206044 | LOC_Os10g43075 |
| control | 11 | qtl11.1 | SNP-11.17221237. | 17707382 | 5.55 | 17702604 | 17705642 | LOC_Os11g30440 |
| | | | | | | 17665368 | 17669580 | LOC_Os11g30400 |
| | | | | | | 17670479 | 17676794 | LOC_Os11g30410 |
| | | | | | | 17694857 | 17696042 | LOC_Os11g30430 |
| | | | | | | 17707270 | 17707530 | LOC_Os11g30450 |
| | | | SNP-11.17705601. | 18191744 | 5.32 | 18150477 | 18154117 | LOC_Os11g31180 |
| | | | | | | 18183621 | 18185946 | LOC_Os11g31200 |
| | | | | | | 18190942 | 18193263 | LOC_Os11g31210 |
| | | | | | | 18171678 | 18174478 | LOC_Os11g31190 |
| control | 12.1 | qtl12.1 | SNP-12.19323008. | 19351539 | 6.57 | 19356955 | 19359845 | LOC_Os12g32090 |
| | | | | | | 19363074 | 19366196 | LOC_Os12g32110 |
| | | | | | | 19369678 | 19376314 | LOC_Os12g32120 |
| | | | | | | 19332567 | 19333950 | LOC_Os12g32040 |
| | | | | | | 19338780 | 19339538 | LOC_Os12g32050 |
| | | | | | | 19346294 | 19347073 | LOC_Os12g32060 |
| | | | | | | 19354159 | 19354899 | LOC_Os12g32080 |
| | | | SNP-12.19391493. | 19420024 | 6.02 | 19405188 | 19408062 | LOC_Os12g32160 |
| | | | SNP-12.19398463. | 19426994 | 6.11 | 19413951 | 19414736 | LOC_Os12g32170 |
| | | | | | | 19424812 | 19428465 | LOC_Os12g32180 |
| | | | | | | 19428927 | 19431251 | LOC_Os12g32190 |
| | | | | | | 19434470 | 19438060 | LOC_Os12g32200 |
| | | | | | | 19440718 | 19443701 | LOC_Os12g32210 |
| | | | SNP-12.19515276. | 19543807 | 6.74 | 19550425 | 19552231 | LOC_Os12g32410 |
| | | | | | | 19552706 | 19556091 | LOC_Os12g32420 |
| | | | | | | 19557622 | 19560049 | LOC_Os12g32430 |
| | | | | | | 19528060 | 19528377 | LOC_Os12g32370 |
| | | | | | | 19531927 | 19534814 | LOC_Os12g32374 |
| | | | | | | 19536726 | 19541133 | LOC_Os12g32380 |
| | | | | | | 19543820 | 19544475 | LOC_Os12g32390 |
| | | | | | | 19546556 | 19547564 | LOC_Os12g32400 |

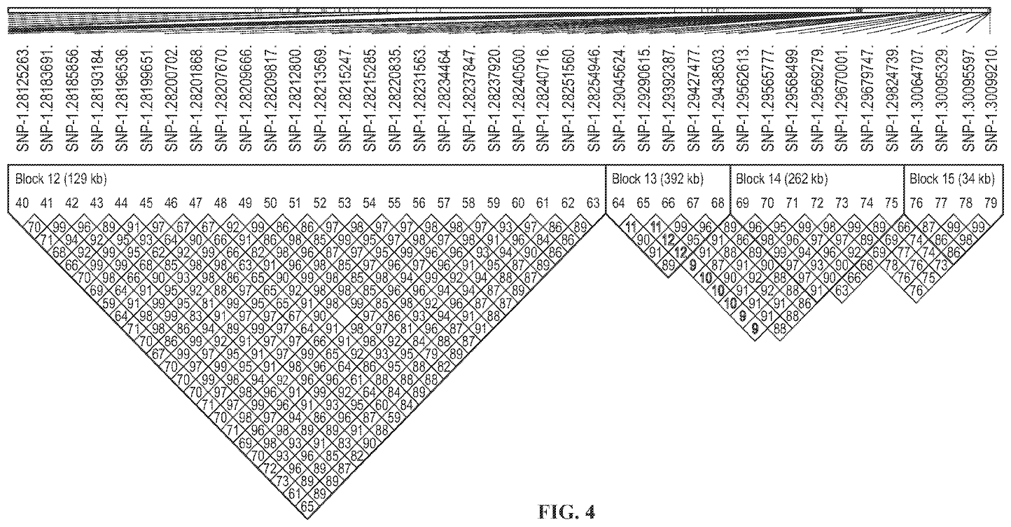

The most significant peak detected for SGW under HNT was located on chr 1 (FIG. 1B). Further analysis showed that this HNT-specific association region encompasses multiple haploblocks (FIG. 4). The largest peak in this region spans from 28186901-28255991 and contains 16 significant SNPs (FIG. 1C). It was found that Big Grain 3 (BG3) (also known as OsPUP4) is located ˜190 kb upstream of SNP-1.28185856. OsPUP4 positively regulates grain size and weight by modulating the long-distance cytokinin transport (Xiao et al., 2019, J. Integr. Plant Biol., 61:581-97).

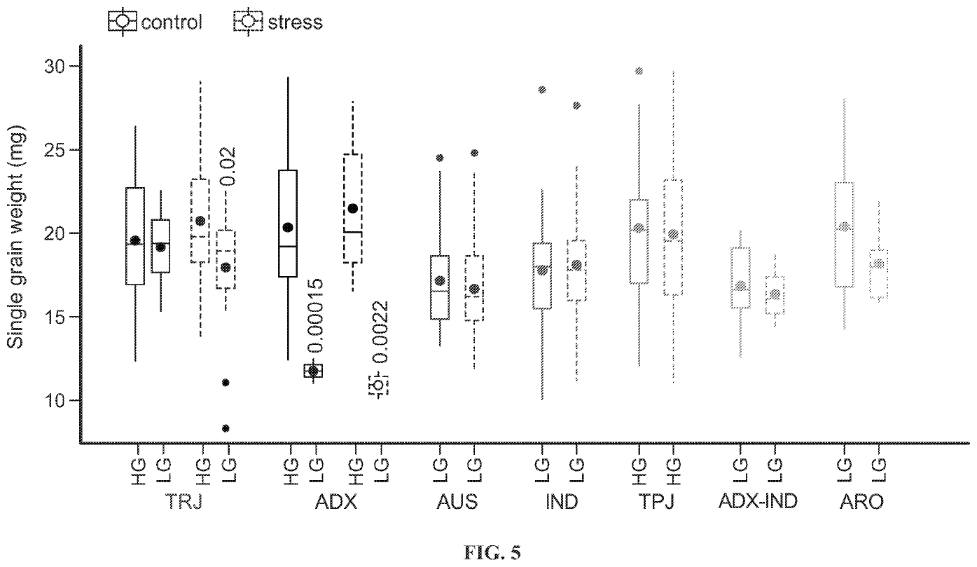

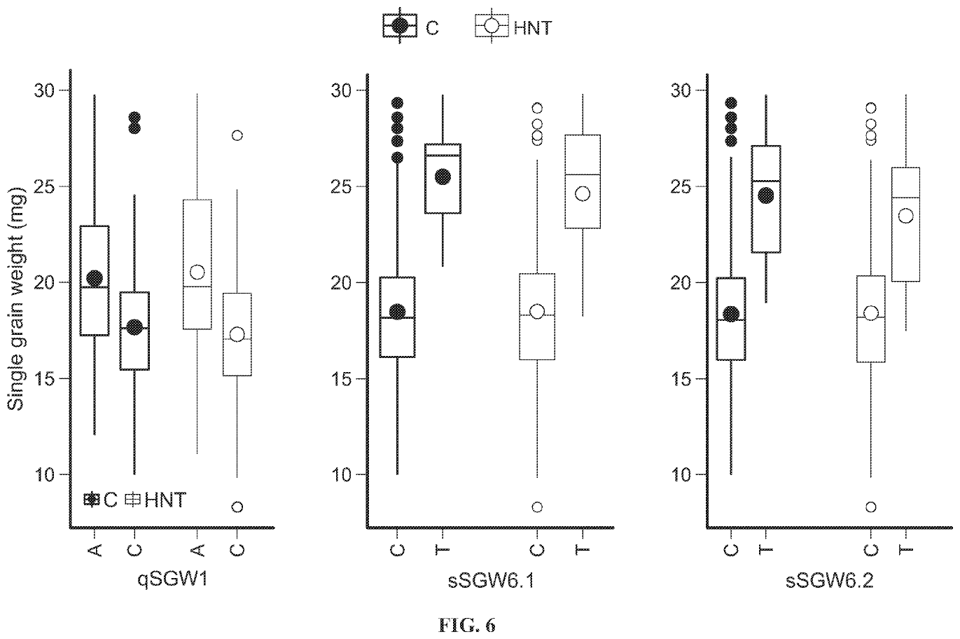

The most significant peak/SNPs for HNT treatment localized near 29 mb (SNP-1.29427477, position=29428523, −log₁₀(P)=6.25; SNP-1.29438503, position=29439549, −log₁₀(P)=6.284). The lead HNT-specific SNP-1.29438503 (hereafter, qSGW1) explained up to 10.5% (R2=0.105) of phenotypic variation under HNT, but was not significant under control (FIG. 1B, 1C). Two major allelic groups for qSGW1 were heavy-grain accessions (HGA), containing the “C” allele, and light-grain accessions (LGA), containing the “A” allele. Considering the entire RDP1 panel, HGA show significantly higher SGW than LGA under both control and HNT (FIG. 1D). It was found that all indica (n^{LGA}=36) and aus (n^{LGA}=38) accessions have the LGA allele and all temperate japonica accessions (n^{HGA}=44) have the HGA allele (FIG. 1E). Tropical japonica has both the LGA (n^{LGA}=18) and HGA (n^{HGA}=35) alleles. Since most indica accessions have higher grain number and lower grain weight than temperate japonica, this could result in a larger difference in SGW between the two allelic groups under both control and HNT (Eizenga et al., 2014, J. Plant Regist., 8:109-16). Given the pre-dominance of one of HGA or LGA allele in both indica and temperate japonica, we decided to examine the allelic effect of HGA and LGA among the tropical japonica accessions. In tropical japonicas, SGW of HGA is significantly higher than LGA but only under HNT stress condition (FIG. 5).

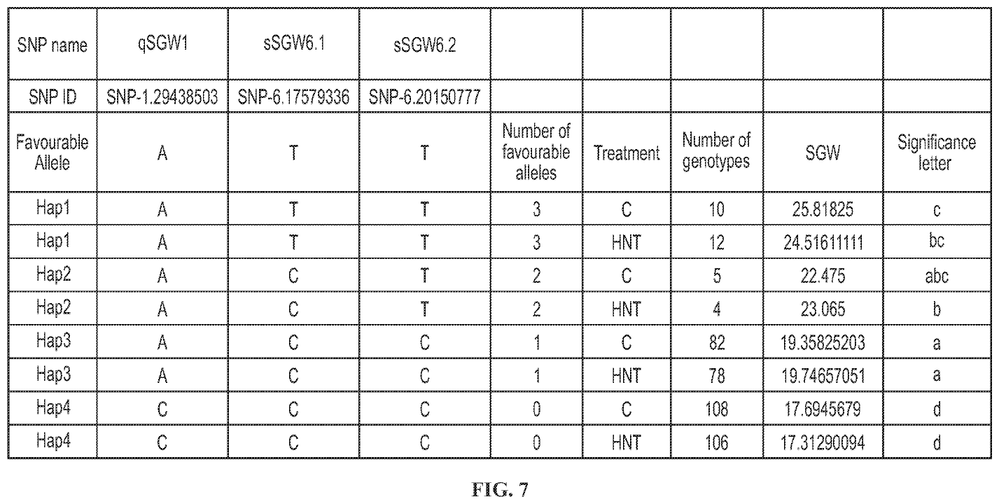

Next, we aimed to determine the SGW outcome for stacking the favorable alleles of the lead HNT-specific SNP (qSGW1) and common (detected under both control and HNT) SNPs on Chr 6 (qSGW6). The markers SNP-1.29438503 (qSGW1), SNP-6.17579336 (sSGW6.1), and SNP-6.20150777 (sSGW6.2) were considered for this analysis, and corresponding favorable alleles were “A”, “T” and “T”, respectively (FIG. 6). We noted that the combination of favorable alleles for sSGW6.1 and sSGW6.2 are present at very low frequency in RDP1 (FIG. 7). Based on allelic combinations, four haplotypes were obtained: hap1, hap2, hap3 and hap4, and their SGW analyzed under control and HNT. Hap1 with all 3 favorable alleles had the highest SGW and lowest frequency in RDP1 (FIG. 1F, FIG. 7). On the contrary, hap4 with all unfavorable alleles had the lowest SGW and highest frequency. Comparing hap 1 (ATT) and hap 3 (ACC) under control and HNT showed that favorable allele (T) for sSGW6.1 and sSGW6.2 showed higher HNT sensitivity than C allele. In contrast, favorable allele (HGA, A) for qSGW1 showed lower sensitivity than the corresponding unfavorable allele, LGA (C). Although SGW for Hap1 decreases in response to HNT, it still maintained higher SGW than the other three haplotypes. These analyses suggest that stacking novel HNT loci has the potential to improve rice resilience to HNT stress for single grain weight.

Since allelic variation at qSGW1 has the most significant effect on SGW under HNT stress, the genes that are in close proximity to the lead SNP were examined. qSGW1 localized to the first intron of a Lonely Guy-like 1 (LOG L1; Os0151210). Some members of the LOG gene family encode for cytokinin activating enzymes that converts inactive cytokinin nucleotides to active free-base forms, N6-(A2-isopentenyl) adenine (iP), trans-zeatin (tZ) (Kuroha et al., 2009, Plant Cell, 21:3152-69). A LOG-family gene, LOG regulates shoot apical meristem (SAM) and inflorescence development in rice (Kurakawa et al., 2007, Nature, 445:652-55). Given this evidence, we decided to test if LOG L1 is the causal gene underlying qSGW1 for SGW under HNT stress.

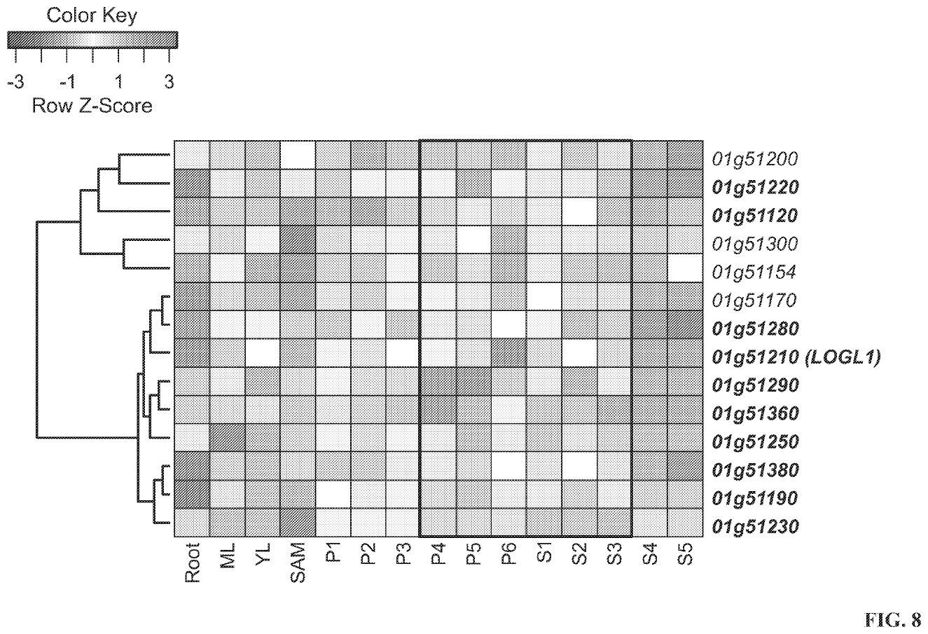

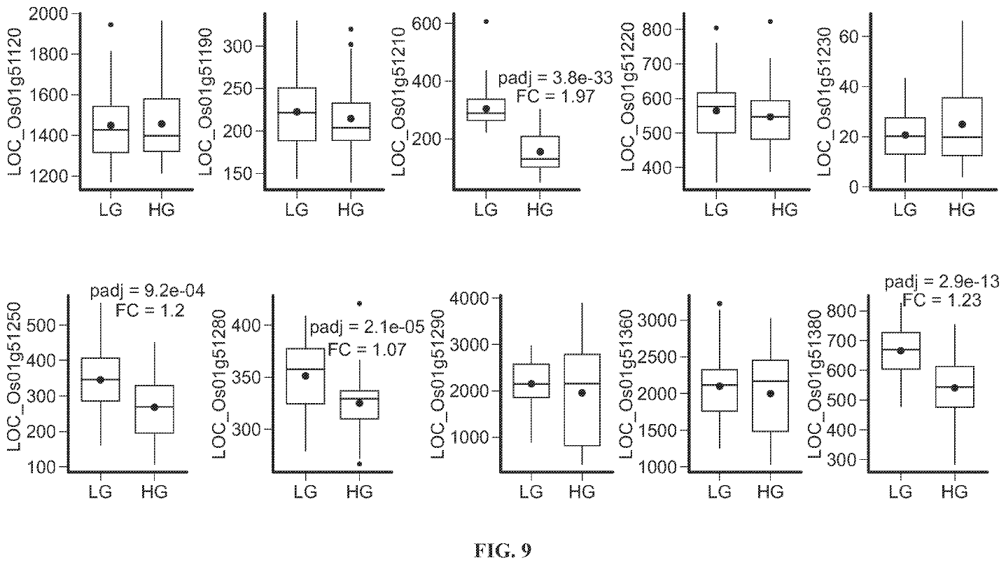

It was determined whether transcript abundance of LOG L1 was different among HGA and LGA haplotypes. Considering 100-200 kb LD decay, we carefully investigated 30 genes within a 100 kb interval of lead SNPs. Out of 30 genes, 6 belong to transposons category, and 10 had very low expression during seed development (Table 4). Given that our HNT treatment spanned the grain development stage of rice, 10 genes that had higher expression during flowering or grain development stage were prioritized (FIG. 8). Among these 10 genes, LOG L1 has very high expression in developing panicles and young seeds near flowering phase (FIG. 8). To assess the allelic variations at transcript level, the natural variation in expression of these 10 genes (including LOG L1) was explored in publicly available RDP1 seedling transcriptome data (GSE 98455; FIG. 9). Notably, LOG L1 showed about 2-fold higher transcript abundance in LGA than HGA. For other genes, expression levels were not significantly (cutoff: pval<0.01 and FC>1.5) different between LGA and HGA. These results suggest that higher transcript abundance of LOG L1 in rice accessions is negatively associated with SGW under HNT stress.

TABLE 4
List of genes located near qSGW1

| | | | | | Transposable | Expressed |
|---|---|---|---|---|---|---|
| Chromosome | MSU Locus D | Gene start | Gene stop | MSU Annotation | element | in seed |
| Chr1 | LOC_Os01g51060 | 29326689 | 29334795 | hydrolase, putative, expressed | no | no |
| Chr1 | LOC_Os01g51110 | 29357538 | 29358055 | expressed protein | no | NA |
| Chr1 | LOC_Os01g51120 | 29364527 | 29368029 | vesicle transport v-SNARE protein, | no | yes |
| | | | | putative, expressed | | |
| Chr1 | LOC_Os01g51140 | 29382534 | 29385653 | helix-loop-helix DNA-binding domain | no | no |
| | | | | containing protein, expressed | | |
| Chr1 | LOC_Os01g51154 | 29405623 | 29411374 | single myb histone, putative, expressed | no | yes |
| Chr1 | LOC_Os01g51170 | 29412196 | 29413972 | glycylpeptide N-tetradecanoyltransferase 1, | no | yes |
| | | | | putative, expressed | | |
| Chr1 | LOC_Os01g51190 | 29423421 | 29428115 | expressed protein | no | ycs |
| Chr1 | LOC_Os01g51200 | 29431601 | 29436466 | CK1_CaseinKinase_1.3 - CK1 includes the | no | yes |
| | | | | casein kinase 1 kinases, expressed | | |
| Chr1 | LOC_Os01g51210 | 29437276 | 29439882 | uncharacterized protein PA4923, putative, | no | yes |
| | | | | expressed | | |
| Chr1 | LOC_Os01g51220 | 29442650 | 29447144 | transport protein particle component, Bet3, | no | yes |
| | | | | domain containing protein, expressed | | |
| Chr1 | LOC_Os01g51230 | 29447990 | 29450454 | IQ calmodulin-binding motif domain | no | yes |
| | | | | containing protein, expressed | | |
| Chr1 | LOC_Os01g51240 | 29454654 | 29455461 | hypothetical protein | no | no |
| Chr1 | LOC_Os01g51250 | 29458644 | 29463035 | mitochondrial carrier protein, putative, | no | yes |
| | | | | expressed | | |
| Chr1 | LOC_Os01g51260 | 29469565 | 29471507 | MYB family transcription factor, putative, | no | no |
| | | | | expressed | | |
| Chr1 | LOC_Os01g51270 | 29475540 | 29475833 | THION44 - Plant thionin family protein | no | NA |
| | | | | precursor, putative, expressed | | |
| Chr1 | LOC_Os01g51280 | 29479108 | 29482865 | 5-nucleotidase surE, putative, expressed | no | yes |
| Chr1 | LOC_Os01g51290 | 29483207 | 29492556 | protein kinase family protein, putative, | no | yes |
| | | | | expressed | | |
| Chr1 | LOC_Os01g51300 | 29501122 | 29506955 | WD domain, G-beta repeat domain | no | yes |
| | | | | containing protein, expressed | | |
| Chr1 | LOC_Os01g51310 | 29508272 | 29509435 | expressed protein | no | no |
| Chr1 | LOC_Os01g51320 | 29513284 | 29514918 | peroxisomal N-acetyl-spermine/spermidine | no | no |
| | | | | oxidase precursor, putative, expressed | | |
| Chr1 | LOC_Os01g51330 | 29521077 | 29523276 | expressed protein | no | no |
| Chr1 | LOC_Os01g51360 | 29529726 | 29532847 | lipase, putative, expressed | no | yes |
| Chr1 | LOC_Os01g51370 | 29534257 | 29536165 | expressed protein | no | NA |
| Chr1 | LOC_Os01g51380 | 29536683 | 29542409 | ATP synthase, putative, expressed | no | yes |
| Chr1 | LOC_Os01g51080 | 29345445 | 29348224 | retrotransposon protein, putative, | yes | NA |
| | | | | unclassified, expressed | | |
| Chr1 | LOC_Os01g51090 | 29348709 | 29351599 | retrotransposon protein, putative, | yes | NA |
| | | | | unclassified, expressed | | |
| Chr1 | LOC_Os01g51100 | 29352683 | 29355515 | retrotransposon protein, putative, | yes | NA |
| | | | | unclassified, expressed | | |
| Chr1 | LOC_Os01g51130 | 29369748 | 29376123 | transposon protein, putative, unclassified, | yes | NA |
| | | | | expressed | | |
| Chr1 | LOC_Os01g51180 | 29416278 | 29423224 | transposon protein, putative, unclassified, | yes | NA |
| | | | | expressed | | |
| Chr1 | LOC_Os01g51340 | 29525181 | 29526530 | retrotransposon protein, putative, | yes | NA |
| | | | | unclassified, expressed | | |

NA: not available

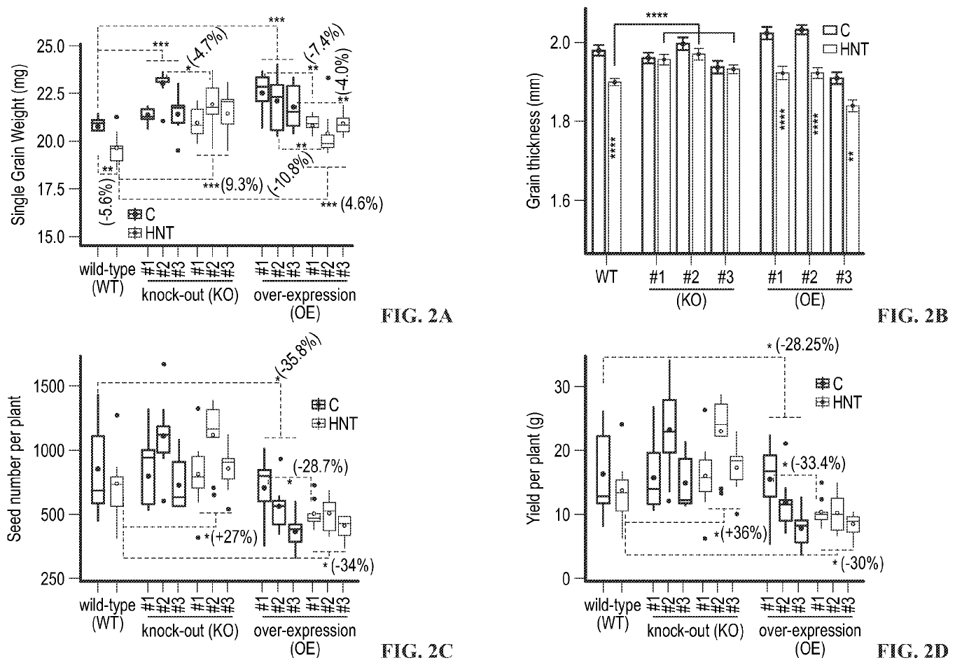

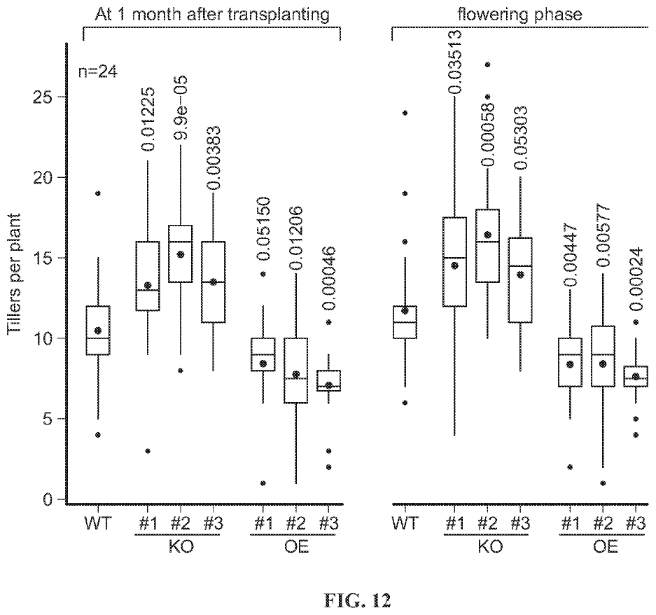

To test association between LOG L1 abundance and HNT grain level outcome, 35S promoter-driven over-expression (OE) and CRISPR-Cas9 (CR)-based knockout (KO) mutants were generated in cv Kitaake, which carries HG allele (A) at qSGW1 locus (Jain et al., 2019, BMC Genomics, 20:1-9) (FIG. 9). Three homozygous KOs targeting two different regions (gR2, #1 and #2; gR1 #3) of LOG L1 had 1 bp deletion for #1, 1 bp insertion for #2 and 41 bp deletion for #3 (FIG. 9B). Both types of mutations resulted in a premature stop codon and a ˜2.5-fold (log 2) reduction in transcript accumulation (FIG. 10). OE lines had 2.5-5 fold (log 2) higher transcript abundance in 2 DAF grains (FIG. 10). We grew all mutants and WT plants for phenotyping under control and HNT stress conditions during grain development. Under control conditions, both KO and OE exhibited higher SGW and seed size compared to WT (FIG. 2A-2B, FIG. 10). Next, the phenotypic response of mutants and WT under HNT treatment was investigated (FIG. 2A-2B, FIG. 10). Compared to corresponding control seeds, OE plants showed maximum reduction (4-10%) in SGW in response to HNT, followed by WT. OE plants also showed significant reduction in grain length, width, area, and thickness. By contrast, SGW of KO lines under HNT was similar to corresponding controls, except reduction for KO #2. Further, KO lines had higher while OE lines had reduced tiller number compared to WT (FIG. 11). These results were consistent with lower seed number, and yield of OE compared to WT and KO lines under control conditions (FIG. 2C-2D). Since OE plants showed reduced seed number compared to WT and KOs and hence, higher SGW OE lines than WT under control could be due to reduced seed number (FIG. 2C). Collectively, these results suggest that LOG L1 regulates multiple yield traits including tiller number, panicle number, and balance between SGW and seed number. It is notable that OE lines showed significantly lower seed number and yield per plant compared to WT under HNT. By contrast, HNT-treated KO lines had significantly higher seed number and yield per plant compared to WT (FIG. 2C-2D). These results suggest that LOG L1 negatively regulates yield traits including seed number and weight under HNT. This is consistent with GWAS findings that LGA varieties with higher LOG L1 transcripts showed lower SGW under HNT. The cell size and number of outer epidermal cells were next investigated and it was found that OE seeds showed significant reduction in cell number per unit area on exposure to HNT compared to corresponding controls (Table 5, FIG. 12).

TABLE 5
Cell size and number analysis of outer epidermal surface of rice mature seeds from wild-type (WT),
overexpression (OE2) and knockout mutants (KO) subjected to control, high night temperature (HNT)

| | | | Number of | Number of | | Cell | Cell | |
|---|---|---|---|---|---|---|---|---|
| | | | cells in | cells in | | Width in | length in | Number |
| | | Number | transverse | longitudinal | | transverse | longitudinal | of cells |
| Genotype | Treatment | of cells | direction | direction | Cell Area | direction | direction | per mm² |
| WT | C | 49.67 ± 2.19^{a} | 6.34 ± 0.34^{a} | 7.85 ± 0.09^{a} | 6086.03 ± 258^{b} | 75.44 ± 3.78^{ab} | 80.75 ± 0.85^{b} | 164.93 ± 7.26^{a} |
| | HNT | 47.67 ± 1.46^{a} | 6.34 ± 0.34^{a} | 7.55 ± 0.25^{a} | 6329.67 ± 195^{b} | 75.44 ± 3.78^{ab} | 84.14 ± 2.75^{b} | 158.29 ± 4.83^{a} |
| CR #2 | C | 50.67 ± 3.85^{a} | 6.5 ± 0.5^{a} | 7.6 ± 0.38^{a} | 6010.07 ± 438^{b} | 74.07 ± 6.18^{b} | 83.83 ± 4.07^{b} | 168.25 ± 12.77^{a} |
| | HNT | 51 ± 2.52^{a} | 6.5 ± 0.5^{a} | 7.33 ± 0.3^{ab} | 5935.17 ± 307^{b} | 73.93 ± 5.29^{b} | 86.74 ± 3.59^{b} | 169.36 ± 8.36^{a} |
| OE #2 | C | 51.34 ± 2.91^{a} | 6.5 ± 0.29^{a} | 7.7 ± 0.17^{a} | 5905.2 ± 342^{b} | 73.41 ± 3.27^{b} | 82.39 ± 1.77^{b} | 170.46 ± 9.65^{a} |
| | HNT | 38.67 ± 2.41^{b} | 5.57 ± 0.35^{a} | 6.75 ± 0.17^{b} | 7853.03 ± 513^{a} | 86.04 ± 5.32^{a} | 94 ± 2.28^{a} | 128.39 ± 7.98^{b} |

Collectively, these results show that LOG L1 is key regulator of single grain weight in rice plants that are exposed to warmer night temperatures during grain development. Among the KO lines generated for this study, we observed that KO #2 had significantly higher SGW under control conditions relative to grain obtained from WT and OE line (FIG. 2A). The higher yield potential for KO #2 is also supported by higher per plant grain weight of this particular gene edited line (FIG. 2D). These data suggest that KO #2 could be a desirable resource for yield increase in rice even under optimal temperature conditions (FIG. 2D). Importantly, KO #2 also performance favorable against other two KO lines for LOG L1 under HNT stress conditions.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. An isolated nucleic acid molecule, wherein the nucleic acid molecule has at least 95% sequence identity to SEQ ID NO:1 or a portion thereof.

2. The isolated nucleic acid molecule of claim 1, wherein the nucleic acid molecule has at least 99% sequence identity to SEQ ID NO:1 or a portion thereof.

3. The isolated nucleic acid molecule of claim 1, wherein the nucleic acid molecule is SEQ ID NO:1 or a portion thereof.

4. A transgenic rice plant transformed with the nucleic acid molecule of claim 1.

5. The transgenic rice plant of claim 4, wherein the nucleic acid molecule is operably linked to a promoter functional in rice plants.

6. A rice plant, or part thereof, comprising a genomic mutation in an endogenous nucleic acid molecule having at least 95% sequence identity to SEQ ID NO:1 and encoding a polypeptide, wherein the genomic mutation confers reduced expression of the endogenous nucleic acid molecule.

7. The rice plant, or part thereof, of claim 6, wherein the nucleic acid molecule has at least 99% sequence identity to SEQ ID NO:1.

8. The rice plant, or part thereof, of claim 6, wherein the nucleic acid molecule is SEQ ID NO:1.

9. The rice plant, or part thereof, of claim 6, wherein the genomic mutation comprises an insertion, a deletion or a substitution.

10. A method of making a mutant rice plant, comprising the steps of:
a) inducing mutagenesis in rice cells;
b) obtaining one or more plants from the cells; and
c) identifying at least one of the plants that contains a mutation in a gene having a wild-type sequence as set forth in SEQ ID NO:1 and encoding a polypeptide that regulates grain weight and/or grain number per plant,
wherein the at least one of the plants that contains the mutation exhibits increased grain weight and/or grain number per plant.

11. The method of claim 10, wherein the rice cells are in a seed.

12. The method of claim 10, further comprising the steps of d) crossing the at least one of the plants that contains the mutation with a second rice plant; and e) selecting progeny of the cross that have the at least one mutation, wherein the progeny plant is homozygous for the at least one mutation.

13. The method of claim 10, further comprising the steps of collecting seed produced by the at least one progeny rice plant.

14. The method of claim 13, further comprising the step of growing a rice plant from the at least one progeny plant from the seed.

15. A method for producing a rice plant comprising the steps of:
a) providing a first rice plant and a second rice plant, the first rice plant having a mutation in an endogenous nucleic acid sequence having a wild-type sequence as set forth in SEQ ID NO:1 and encoding a polypeptide that regulates grain weight and/or grain number per plant, wherein the first plant exhibits higher grain weight under nighttime or daytime temperature stress, wherein the second plant contains a desired phenotypic trait;
b) crossing the first rice plant with the second rice plant to produce one or more F1 progeny plants;
c) collecting seed produced by the F1 progeny plants; and
d) germinating the seed to produce rice plants exhibiting higher grain weight under nighttime or daytime temperature stress.

16. The method of claim 15, wherein the desired phenotypic trait is selected from the group consisting of disease resistance; high yield; mechanical harvestability; maturation; and grain number per plant.

17. The method of claim 15, further comprising the steps of collecting seed produced by the at least one progeny plant.

18. The method of claim 17, further comprising the steps of growing a plant from the at least one progeny plant from the seed.


SEQUENCE LISTING (28 sequences)

SEQ ID NO: 1 - DNA, 672 bp, Oryza sativa
  source 1..672: mol_type = genomic DNA
atgggcgacaacagcgccgccgcggcggccgtggccgcgccgcgcggcaggttcggcaggatctgcgtcttctgcggcagcaacgccggcaaccgcgcggtgttcggcgacgcggcgctccagctcgggcaggagctggtgtcgagagggatcgagttggtctacggtggcggcagcgtcgggttgatgggcttgatcgcgcagacggttcttgatggcggctgcggtgttctcggggtgattccaaaagcactcatgcccaccgagatatcaggtgcaagtgttggagaagtgaaaattgtgtctgacatgcatgagaggaaagctgagatggcacgccaatccgatgccttcatcgctcttcctggagggtatggaacaatggaggagttgttagagatgataacttggtcacaacttggaattcatgacaaaccagttgggttgctgaatgtggacggttactatgatccgttgcttgcgctatttgataagggtgcggcagaaggatttattaaggccgattgcagacaaataattgtttcggcaccgactgcgcatgagctgctgagaaagatggagcaatacactcgttcacaccaggaggtagcgccacgtacaagctgggagatgtcagagcttggttatggaaagacaccagaggaatcgtag

SEQ ID NO: 2 - protein, 223 aa, Oryza sativa
MGDNSAAAAAVAAPRGRFGRICVFCGSNAGNRAVFGDAALQLGQELVSRGIELVYGGGSVGLMGLIAQTVLDGGCGVLGVIPKALMPTEISGASVGEVKIVSDMHERKAEMARQSDAFIALPGGYGTMEELLEMITWSQLGIHDKPVGLLNVDGYYDPLLALFDKGAAEGFIKADCRQIIVSAPTAHELLRKMEQYTRSHQEVAPRTSWEMSELGYGKTPEES

SEQ ID NO: 3 - DNA, 672 bp, Oryza sativa
  source 1..672: mol_type = genomic DNA
atgggagacggagcggaggcggcgggggcgaccgcggcgagcaggttcgggacgatctgcgtcttctgcggcagcaacgcggggcgccgcagggtgttcggcgacgcggcgctcgacctcggccacgagctggtgaggcggggcgtcgatctggtctacggcggcggcagcatcgggctgatgggcttgatcgcgcgtacggttctcgacggcggccgccgtgtcgtcggggtgattcctagagctctcatggctgtcgagatatcaggtgagagtgtgggagaagtaatagttgtacaggacatgcatgagcggaaagcggagatggctcggcgatccaaagcgttcattgctcttcctgggggctatggaacaatggaggagctgttagagatgataacatggtgccaacttggaattcatgacaagccagttggattgctaaatgttgacggttactacgatccactgctcgcgttgttcgacaaaggcgaggcggagggcttcatcaactctgattgtagacaaatatttgtatctgcaccaactgcaagtgaattgctgacaaagatggagcaatacactcggttgcaccaggaggtggcccctgcaacaagctgggagatctcagagcttggctatggaagaacaccgggcgctgatcaatcctag

SEQ ID NO: 4 - protein, 223 aa, Oryza sativa
MGDGAEAAGATAASRFGTICVFCGSNAGRRRVFGDAALDLGHELVRRGVDLVYGGGSIGLMGLIARTVLDGGRRVVGVIPRALMAVEISGESVGEVIVVQDMHERKAEMARRSKAFIALPGGYGTMEELLEMITWCQLGIHDKPVGLLNVDGYYDPLLALFDKGEAEGFINSDCRQIFVSAPTASELLTKMEQYTRLHQEVAPATSWEISELGYGRTPGADQS

SEQ ID NO: 5 - DNA, 657 bp, Zea mays
  source 1..657: mol_type = genomic DNA
atgggcgattccgcggcgggcgcgccggagccgagcaggttcggcaggatctgcgtcttctgcggcagcaaccccggcaatcgcgccgtctacggggacgcagcgctcgacctcggcaaagagctggtggcgaaggggatcgatttggtctacggcggcgggagcgtcgggctcatgggcctgatcgcgcagacggtccttggtggcggctgcagtgtcctcggagtgattccgagagcactcatgccgcttgagatatctggtgctagcgttggagaagttaaggttgtctccgacatgcacgagaggaaagctgagatggcacggcaagctgatgccttcatagctcttcctggagggtacggaacaatggaagaactgttagagatgataacgtggtcacagctcggaattcatgacaaaccagtcggtttgctgaacgttgatggctactacgacccgctgctcatgctgtttgatagaggggcaacggaaggctttatcaagctagattgcagagatataattgtttcagcgccaactgcccatgagttactgaagaagatggagcactacactcggtcgcaccaggaggtcgccccacggacgagctgggagatgtcagagctgggctatggaaaagcgtcggagtcatag

SEQ ID NO: 6 - protein, 218 aa, Zea mays
MGDSAAGAPEPSRFGRICVFCGSNPGNRAVYGDAALDLGKELVAKGIDLVYGGGSVGLMGLIAQTVLGGGCSVLGVIPRALMPLEISGASVGEVKVVSDMHERKAEMARQADAFIALPGGYGTMEELLEMITWSQLGIHDKPVGLLNVDGYYDPLLMLFDRGATEGFIKLDCRDIIVSAPTAHELLKKMEHYTRSHQEVAPRTSWEMSELGYGKASES

SEQ ID NO: 7 - DNA, 834 bp, Triticum aestivum
  source 1..834: mol_type = genomic DNA
atgccgccttcatttgaagcttctcccccagatactctacgagataacacacggcatcgggtagaagaacgaaccgaacccaaacacctctcctccacgacgactcttaaccccatcctcccgccccgcacccgagaggaaacaagccaggccatgggcgacaccgccgcgcccgcggccgcgccgccgaggaagttcggcaggatctgcgtcttctgcggcagcaactccggcaatcgcgcggtgttcggcgacacggcgctcgagctcggccaggggctggtgacgaggggggttgatctggtctatggcgggggcagcatcgggttgatgggcctgatcgcgcagacggttctggatggcggctgccgtgtcctcggggtgattcccagagcactcatgccccttgagatctctggtgcaagtgttggagaagtaaagatagtctctgacatgcatgagaggaaagctgagatggcgcgacaagctgatgcattcattgctcttccgggagggtatggaacaatggaggagttgttagagatgatcacatggtcgcagctcggaatccatgacaaaccagttggcttgctaaacgtcgacgggtactatgatccgttacttgcgctgttcgacaagggcgcggcagaaggttttattaaggccgattgcaggcagataatcgtgtcggcgccaactgcccacgaactgctgacgaaaatggagcaatacacccgttcacaccgggaggtggcgtcgcggacgagctgggagatgaccgagctgggctacgggaaagctgcaccagagccggaggaggaggcgtcgtgctga

SEQ ID NO: 8 - protein, 277 aa, Triticum aestivum
MPPSFEASPPDTLRDNTRHRVEERTEPKHLSSTTTLNPILPPRTREETSQAMGDTAAPAAAPPRKFGRICVFCGSNSGNRAVFGDTALELGQGLVTRGVDLVYGGGSIGLMGLIAQTVLDGGCRVLGVIPRALMPLEISGASVGEVKIVSDMHERKAEMARQADAFIALPGGYGTMEELLEMITWSQLGIHDKPVGLLNVDGYYDPLLALFDKGAAEGFIKADCRQIIVSAPTAHELLTKMEQYTRSHREVASRTSWEMTELGYGKAAPEPEEEASC

SEQ ID NO: 9 - DNA, 675 bp, Hordeum vulgare
  source 1..675: mol_type = genomic DNA
atgggcgacaccaccgcgccctcgccgccgaggaggttcggcaggatctgcgtcttctgcggcagcaactccggcaaccgcgccgtgttcggcgacgccgcgctcgagctcggccagggcctggtgacgaggggggtcgatctggtctacggcggcggcagcatcgggctgatgggcctgatcgcgcagacggttctcgacggcggctgccgcgtcctcggggtgattccaagagcactcatgcccctcgagatatccggtgcaagtgttggagaagtaaagattgtctccgacatgcatgagaggaaagctgagatggcgcgacaagccgatgcattcattgctcttccgggtgggtatggaacaatggaagagctgttagagatgatcacttggtcgcagcttggaatccatgacaaaccggtcgggttgctaaacgtcgatgggtactatgatccgttactcgcgctgttcgacaagggcgcggcggaagggtttattaaggccgattgcaggcagataatcgtgtcggcaccaactgcccacgaactgctgacaaaaatggagcaatacacccgttcacaccgggaggtggcctcgcggacgagctgggagatgaccgagatgggctacgggaaagcaccggagccggaggaggaggcggcggcatcgtag

SEQ ID NO: 10 - protein, 224 aa, Hordeum vulgare
MGDTTAPSPPRRFGRICVFCGSNSGNRAVFGDAALELGQGLVTRGVDLVYGGGSIGLMGLIAQTVLDGGCRVLGVIPRALMPLEISGASVGEVKIVSDMHERKAEMARQADAFIALPGGYGTMEELLEMITWSQLGIHDKPVGLLNVDGYYDPLLALFDKGAAEGFIKADCRQIIVSAPTAHELLTKMEQYTRSHREVASRTSWEMTEMGYGKAPEPEEEAAAS

SEQ ID NO: 11 - DNA, 879 bp, Sorghum bicolor
  source 1..879: mol_type = genomic DNA
atgtgggctcctgtggtgcaatctggtgcggcactggagttgacccggcaaattgattacgcgattattaaacagcacgatcgcgtcatgagagaagccctgatgccgccttcatttgaagctcccccgtcccgtcccctctcctcgaaggcaaagaagccaaagccgaacacctcactttatccgccgtcccgccgccactgcccgctccgctcctccgccatgggcgacaccgtagcggacgcgccggagccgagcaggttcggcaggatctgcgtcttctgcggcagcaaccccggcaatcgcgccgtctacggggacgccgcgctcgacctcggcaaagagctggtggcgagggggatcgatttggtctacggcggcggcagcgtcgggctcatgggcctgatcgcgcagacggttcttgacggcggctgcagtgtcctaggggtgattccaagagcactcatgccgcttgagatatctggtgctagcgttggagaagtaaaggttgtctccgatatgcacgaaagaaaagctgagatggcgcgacaagctgatgccttcattgctcttcctggagggtatggaacaatggaagagctgttagagatgataacatggtcacaactaggaattcatgataaaccagttggattactaaatgttgatggttactacgacccgttgctcatgctatttgacaaaggggcgacggaaggttttattaagctagattgcagagatataattgtttcagcaccaactgcccatgaattgctggagaaaatggagcactacactcggtcacaccaggaggttgccccacggacgagctgggagatgtcagagctgggctatggaaaagcaccggagtcatag

SEQ ID NO: 12 - protein, 292 aa, Sorghum bicolor
MWAPVVQSGAALELTRQIDYAIIKQHDRVMREALMPPSFEAPPSRPLSSKAKKPKPNTSLYPPSRRHCPLRSSAMGDTVADAPEPSRFGRICVFCGSNPGNRAVYGDAALDLGKELVARGIDLVYGGGSVGLMGLIAQTVLDGGCSVLGVIPRALMPLEISGASVGEVKVVSDMHERKAEMARQADAFIALPGGYGTMEELLEMITWSQLGIHDKPVGLLNVDGYYDPLLMLFDKGATEGFIKLDCRDIIVSAPTAHELLEKMEHYTRSHQEVAPRTSWEMSELGYGKAPES

SEQ ID NO: 13 - DNA, 25 bp, synthetic construct
  source 1..25: mol_type = other DNA
gtgtgagagggatcgagttggtcta

SEQ ID NO: 14 - DNA, 25 bp, synthetic construct
  source 1..25: mol_type = other DNA
aaactagaccaactcgatccctctc

SEQ ID NO: 15 - DNA, 25 bp, synthetic construct
  source 1..25: mol_type = other DNA
gtgtgaaaagcactcatgcccaccg

SEQ ID NO: 16 - DNA, 25 bp, synthetic construct
  source 1..25: mol_type = other DNA
aaaccggtgggcatgagtgcttttc

SEQ ID NO: 17 - DNA, 22 bp, synthetic construct
  source 1..22: mol_type = other DNA
caccatgggcgacaacagcgcc

SEQ ID NO: 18 - DNA, 27 bp, synthetic construct
  source 1..27: mol_type = other DNA
cgattcctctggtgtctttccataacc

SEQ ID NO: 19 - DNA, 22 bp, synthetic construct
  source 1..22: mol_type = other DNA
caccatgggcgacaacagcgcc

SEQ ID NO: 20 - DNA, 21 bp, synthetic construct
  source 1..21: mol_type = other DNA
tacgtgatagacccttgtgcc

SEQ ID NO: 21 - DNA, 22 bp, synthetic construct
  source 1..22: mol_type = other DNA
cacagaaaagagaagctgccaa

SEQ ID NO: 22 - DNA, 20 bp, synthetic construct
  source 1..20: mol_type = other DNA
cgccatcaagaaccgtctgc

SEQ ID NO: 23 - DNA, 22 bp, synthetic construct
  source 1..22: mol_type = other DNA
caccatgggcgacaacagcgcc

SEQ ID NO: 24 - DNA, 20 bp, synthetic construct
  source 1..20: mol_type = other DNA
cgggttgatgggcttgatcg

SEQ ID NO: 25 - DNA, 20 bp, synthetic construct
  source 1..20: mol_type = other DNA
ccgttgcttgcgctatttga

SEQ ID NO: 26 - DNA, 20 bp, synthetic construct
  source 1..20: mol_type = other DNA
atctcccagcttgtacgtgg

SEQ ID NO: 27 - DNA, 20 bp, synthetic construct
  source 1..20: mol_type = other DNA
tcaacgtggagaagcagatg

SEQ ID NO: 28 - DNA, 20 bp, synthetic construct
  source 1..20: mol_type = other DNA
atggcagtgatagcctgctt